(19) United States
(12) Patent Application Publication
(10) Pub. No.: US 2026/0207863 A1
(43) Pub. Date: Jul. 23, 2026

(54) MIST INHALATION POD FOR DELIVERING A THERAPEUTIC

(71) Applicant: SHAHEEN INNOVATIONS HOLDING LIMITED, Abu Dhabi (AE)

(72) Inventors: Mohammed Alshaiba Saleh Ghannam Almazrouei, Abu Dhabi (AE); Dinil Divakaran, Abu Dhabi (AE); Clement Lamoureux, Abu Dhabi (AE); Jeff Machovec, Rochester, MN (US); Peter Lomas, Wirral (GB); Nveed Chaudhary, Southampton (GB)

(21) Appl. No.: 19/530,078

(22) Filed: Feb. 4, 2026

Related U.S. Application Data
(63) Continuation-in-part of application No. 19/254,926, filed on Jun. 30, 2025.
(60) Provisional application No. 63/679,375, filed on Aug. 5, 2024.
(60) Provisional application No. 63/928,635, filed on Dec. 1, 2025.

(51) Int. Cl. A61M 15/00; A24F 40/10

(52) U.S. Cl. CPC A61M 15/001; A24F 40/10; A61M 15/0021; A61M 15/0085; A61M 15/0086; A61M 15/0095

(57) ABSTRACT

A mist inhaler pod for use with a driver. The pod comprises a housing, a liquid barrier wall positioned within the housing having at least one liquid channel, and a liquid chamber defined by the liquid barrier wall and the housing. The pod further includes a spacer positioned within the housing, and a fluid flow manifold provided within the spacer. A sonication chamber is included within a cavity of the fluid flow manifold. An ultrasonic transducer is in communication with the sonication chamber, and a capillary conducts liquid from the liquid chamber to the sonication chamber. An air inlet conduit forms an air-tight channel for conducting air form an air inlet to the sonication chamber, and a mist outlet conduit conducts mist from the sonication chamber to the mist outlet port.

### CROSS REFERENCES TO RELATED APPLICATIONS

The present application claims the benefit of priority to U.S. provisional application no. 63/928635, filed on Dec. 1, 2025. The present application is also a continuation-in-part of U.S. application Ser. No. 19/254926, filed on Jun. 30, 2025 which itself claims the benefit of priority to U.S. provisional application no. 63/679375, filed Aug. 5, 2024, all of which are incorporated by reference herein in their entirety.

### FIELD

The present invention relates to a mist inhalation pod for use with a driver. The present invention more particularly relates to a mist inhalation pod for delivering a therapeutic for inhalation by a user.

### BACKGROUND

Mist inhalers are used for generating a mist or vapour for inhalation by a user. The mist may contain a drug or medicine which is inhaled by a user and absorbed into the user's blood stream.

In particular, mist inhalers or electronic vaporising inhalers are becoming popular among smokers who want to avoid the tar and other harsh chemicals associated with traditional cigarettes and who wish to satisfy the craving for nicotine. Electronic vaporising inhalers may contain liquid nicotine, which is typically a mixture of nicotine oil, a solvent, water, and often flavouring. When the user draws, or inhales, on the electronic vaporising inhaler, the liquid nicotine is drawn into a vaporiser where it is heated into a vapour. As the user draws on the electronic vaporising inhaler, the vapour containing the nicotine is inhaled. Such electronic vaporising inhalers may have medical purpose.

Electronic vaporising inhalers and other vapour inhalers typically have similar designs. Most electronic vaporising inhalers feature a liquid nicotine reservoir with an interior membrane, such as a capillary element, typically cotton, that holds the liquid nicotine so as to prevent leaking from the reservoir. Nevertheless, these cigarettes are still prone to leaking because there is no obstacle to prevent the liquid from flowing out of the membrane and into the mouthpiece. A leaking electronic vaporising inhaler is problematic for several reasons. As a first disadvantage, the liquid can leak into the electronic components, which can cause serious damage to the device. As a second disadvantage, the liquid can leak into the electronic vaporising inhaler mouthpiece, and the user may inhale the unvapourised liquid.

Electronic vaporising inhalers are also known for providing inconsistent doses between draws. The aforementioned leaking is one cause of inconsistent doses because the membrane may be oversaturated or undersaturated near the vaporiser. If the membrane is oversaturated, then the user may experience a stronger than desired dose of vapour, and if the membrane is undersaturated, then the user may experience a weaker than desired dose of vapour. Additionally, small changes in the strength of the user's draw may provide stronger or weaker doses. Inconsistent dosing, along with leaking, can lead to faster consumption of the vaping liquid.

Additionally, conventional electronic vaporising inhalers tend to rely on inducing high temperatures of a metal heating component configured to heat a liquid in the e-cigarette, thus vaporising the liquid that can be breathed in. Problems with conventional electronic vaporising inhalers may include the possibility of burning metal and subsequent breathing in of the metal along with the burnt liquid. In addition, some may not prefer the burnt smell caused by the heated liquid.

Thus, a need exists in the art for improved mist inhalers which seek to address at least some of the problems described herein.

### BRIEF DESCRIPTION OF THE INVENTION

The present invention provides a mist inhalation pod for use with a driver as claimed in claim 1, a mist inhalation pod for use with a driver as claimed in claim 9, and a mist inhalation device as claimed in claim 19. Preferred features of the invention are provided in the dependent claims.

The various examples of this disclosure are described below and have multiple advantages and benefits over conventional vaporising inhalers. These advantages and benefits are set out in the description below.

### REPRESENTATIVE FEATURES

Representative features are set out in the following clauses, which stand alone or may be combined, in any combination, with one or more features disclosed in the text and/or drawings of the specification.

In some examples, there is provided a mist inhalation pod for use with a driver, the pod comprising: a housing having a first end, an opposite second end and at least one side wall extending between the first end and the second end; a first end wall proximate the first end and the side wall, the first end wall closing the first end of the housing, the first end wall being provided with a mist outlet port; a second end wall proximate the second end and the side wall, the second end wall closing the second end of the housing; a liquid barrier wall positioned within the housing and spaced apart from the first end wall, the liquid barrier wall extending towards the side wall of the housing to form a liquid seal between the liquid barrier wall and the side wall, the liquid barrier wall having at least one liquid channel with a liquid inlet and a liquid outlet; a liquid chamber defined by the liquid barrier wall, the first end wall and the side wall of the housing, the liquid chamber containing a liquid to be atomised, the liquid comprising a therapeutic, the liquid inlet being in liquid communication with the liquid chamber for conducting the liquid through the liquid outlet; a spacer positioned within the housing between the liquid barrier wall and the second end wall, the spacer having a perimeter extending toward the side wall of the housing, the spacer including a hollow interior surrounded by the perimeter; a fluid flow manifold positioned at least partially within the hollow interior of the spacer and including a first side proximate the first end wall, and a second side proximate the second end wall, the first side of the fluid flow manifold including a channel having a first portion and a second portion, the first portion extending to the second portion tangentially, and the second side of the fluid flow manifold having a cavity, the cavity including a first aperture for allowing fluid flow in a first direction, and one or more further apertures for allowing fluid flow in a second direction; a sonication chamber including the cavity of the fluid flow manifold; an ultrasonic transducer positioned between the sonication chamber and the second end wall, the ultrasonic transducer having an atomisation surface adjacent to the sonication chamber and in communication with the sonication chamber; a capillary having a first portion at least partly superimposed on the atomisation surface of the ultrasonic transducer and a second portion adjacent to the liquid outlet of the liquid channel, wherein the second portion of the capillary covers at least a portion of the liquid outlet and is configured to conduct the liquid from the liquid outlet to the atomisation surface to generate a mist; an air inlet conduit, including the one or more further apertures in the fluid flow manifold, forming an air-tight channel for conducting air through the spacer and along the channel in the first side of the fluid flow manifold, the air inlet conduit extending from proximate the second end wall, through the spacer and through the fluid flow manifold to the sonication chamber, a first end of the air inlet conduit being in fluid communication with an air inlet port proximate the second end wall of the housing and a second end of the air inlet conduit being in fluid communication with the sonication chamber; and a mist outlet conduit, including the first aperture in the fluid flow manifold, forming an air-tight channel for conducting the mist through the liquid chamber and any liquid contained therein, the mist outlet conduit extending from the first end wall, through the liquid chamber, through the liquid barrier wall, and through the fluid flow manifold to the sonication chamber, a first end of the mist outlet conduit being in fluid communication with a mist outlet port in the first end wall of the housing and a second end of the mist outlet conduit being in fluid communication with the sonication chamber, wherein, in use, the capillary conducts the liquid from the liquid chamber to the atomisation surface to be atomised, and the mist generated is conducted through the mist outlet conduit to the mist outlet port.

In some examples, the first portion of the channel is substantially straight between the spacer and the second portion of the channel.

In some examples the second portion of the channel is annular, the annular second portion of the channel surrounding the first aperture of the fluid flow manifold.

In some examples, the one or more further apertures include at least four apertures, the at least four apertures being spaced around the first aperture, the fluid flow in the first direction thereby being coaxial to the fluid flow in the second direction.

In some examples, the one or more further apertures are slots of the same width and shape as the second portion of the channel.

In some examples, the one or more further apertures of the air inlet conduit are positioned radially inward of the first portion of the capillary.

In some examples, air enters the sonication chamber transverse to the atomisation surface of the ultrasonic transducer.

In some examples, a mouthpiece positioned proximate the first end of the housing, the mouthpiece including a mist inhalation port in fluid communication with the mist outlet port in the housing.

In some examples, there is provided a mist inhalation pod for use with a driver, the pod comprising: a housing having a first end, an opposite second end and at least one side wall extending between the first end and the second end; a first end wall proximate the first end and the side wall, the first end wall closing the first end of the housing, the first end wall being provided with a mist outlet port; a second end wall proximate the second end and the side wall, the second end wall closing the second end of the housing; a liquid barrier wall positioned within the housing and spaced apart from the first end wall, the liquid barrier wall having at least one liquid channel with a liquid inlet and a liquid outlet; a liquid chamber partially defined by the liquid barrier wall, the liquid chamber containing a liquid to be atomised, the liquid comprising a therapeutic, the liquid inlet being in liquid communication with the liquid chamber for conducting the liquid through the liquid outlet; a spacer positioned within the housing between the liquid barrier wall and the second end wall, the spacer including a hollow interior surrounded by a perimeter; a fluid flow manifold positioned at least partially within the hollow interior of the spacer and including a first side proximate the first end wall, and a second side proximate the second end wall, the first side of the fluid flow manifold including a channel having a first portion and a second portion, the first portion extending to the second portion tangentially, and the second side of the fluid flow manifold having a cavity, the cavity including a first aperture for allowing fluid flow in a first direction, and one or more further apertures for allowing fluid flow in a second direction; a sonication chamber including the cavity of the fluid flow manifold; an air inlet conduit for conducting air to the sonication chamber; and a mist outlet conduit for conducting mist to the mist outlet port, wherein, in use, the liquid is conducted from the liquid chamber to the sonication chamber to be atomised, and the mist generated is conducted through the mist outlet port.

In some examples, the first portion of the channel is substantially straight between the spacer and the second portion of the channel.

In some examples, the second portion of the channel is annular, the annular second portion of the channel surrounding the first aperture in the fluid flow manifold.

In some examples, the one or more further apertures include at least four apertures, the at least four apertures being spaced around the first aperture, the fluid flow in the first direction thereby being coaxial to the fluid flow in the second direction.

In some examples, the one or more further apertures are slots of the same width and shape as the second portion of the channel.

In some examples, the one or more further apertures of the air inlet conduit are positioned radially inward of the first portion of the capillary.

In some examples, air enters the sonication chamber transverse to the atomisation surface of the ultrasonic transducer.

In some examples, a mouthpiece positioned proximate the first end of the housing, the mouthpiece including a mist inhalation port in fluid communication with the mist outlet port in the housing.

In some examples, an ultrasonic transducer is positioned between the sonication chamber and the second end wall, the ultrasonic transducer having an atomisation surface adjacent to the sonication chamber and in communication with the sonication chamber.

In some examples, the liquid barrier wall extends towards the side wall of the housing to form a liquid seal between the liquid barrier wall and the side wall.

In some examples, there is provided a mist inhalation device comprising a pod and a driver, the pod comprising: a housing having a first end, an opposite second end and at least one side wall extending between the first end and the second end; a first end wall proximate the first end and the side wall, the first end wall closing the first end of the housing, the first end wall being provided with a mist outlet port; a second end wall proximate the second end and the side wall, the second end wall closing the second end of the housing; a liquid barrier wall positioned within the housing and spaced apart from the first end wall, the liquid barrier wall having at least one liquid channel with a liquid inlet and a liquid outlet; a liquid chamber partially defined by the liquid barrier wall, the liquid chamber containing a liquid to be atomised, the liquid comprising a therapeutic, the liquid inlet being in liquid communication with the liquid chamber for conducting the liquid through the liquid outlet; a spacer positioned within the housing between the liquid barrier wall and the second end wall, the spacer including a hollow interior surrounded by a perimeter; a fluid flow manifold positioned at least partially within the hollow interior of the spacer and including a first side proximate the first end wall, and a second side proximate the second end wall, the first side of the fluid flow manifold including a channel having a first portion and a second portion, the first portion extending to the second portion tangentially, and the second side of the fluid flow manifold having a cavity, the cavity including a first aperture for allowing fluid flow in a first direction, and one or more further apertures for allowing fluid flow in a second direction; a sonication chamber including the cavity of the fluid flow manifold; an air inlet conduit for conducting air to the sonication chamber; and a mist outlet conduit for conducting mist to the mist outlet port, wherein, in use, the liquid is conducted from the liquid chamber to the sonication chamber to be atomised, and the mist generated is conducted through the mist outlet port, and the driver including: a housing having a cavity configured to receive at least part of the pod; and driver circuitry for supplying a drive signal to the pod.

In some examples, the driver housing includes a hole, and the driver includes a conduit extending from the hole to the pod, the conduit configured to convey air from the surroundings to the air inlet conduit of the pod.

In some examples, there is provided a mist inhalation pod for use with a driver, the pod comprising: a housing having a first end, an opposite second end and at least one side wall extending between the first end and the second end; a first end wall proximate the first end and the side wall, the first end wall closing the first end of the housing, the first end wall being provided with a mist outlet port; a second end wall proximate the second end and the side wall, the second end wall closing the second end of the housing; a liquid barrier wall positioned within the housing and spaced apart from the first end wall, the liquid barrier wall having at least one liquid channel with a liquid inlet and a liquid outlet; a liquid chamber partially defined by the liquid barrier wall, the liquid chamber containing a liquid to be atomised, the liquid comprising a therapeutic, the liquid inlet being in liquid communication with the liquid chamber for conducting the liquid through the liquid outlet; a spacer positioned within the housing between the liquid barrier wall and the second end wall, the spacer including a hollow interior surrounded by a perimeter; fluid flow manifold positioned at least partially within the hollow interior of the spacer and including a first side proximate the first end wall, and a second side proximate the second end wall, the first side of the fluid flow manifold including a channel, and the second side of the fluid flow manifold having a cavity, the cavity including a first aperture for allowing fluid flow in a first direction, and one or more further apertures for allowing fluid flow in a second direction; a sonication chamber including the cavity of the fluid flow manifold; an air inlet conduit for conducting air to the sonication chamber; and a mist outlet conduit for conducting mist to the mist outlet port, wherein, in use, the liquid is conducted from the liquid chamber to the sonication chamber to be atomised, and the mist generated is conducted through the mist outlet port.

A mist inhalation pod for use with a driver, the pod comprising: a housing having a first end, an opposite second end and at least one side wall extending between the first end and the second end; a first end wall proximate the first end and the side wall, the first end wall closing the first end of the housing, the first end wall being provided with a mist outlet port; a second end wall proximate the second end and the side wall, the second end wall closing the second end of the housing; a liquid barrier wall positioned within the housing and spaced apart from the first end wall, the liquid barrier wall having at least one liquid channel with a liquid inlet and a liquid outlet; a liquid chamber partially defined by the liquid barrier wall, the liquid chamber containing a liquid to be atomised, the liquid comprising a therapeutic, the liquid inlet being in liquid communication with the liquid chamber for conducting the liquid through the liquid outlet; a spacer positioned within the housing between the liquid barrier wall and the second end wall, the spacer including a hollow interior surrounded by a perimeter; a fluid flow manifold positioned at least partially within the hollow interior of the spacer and including a first side proximate the first end wall, and a second side proximate the second end wall, the first side of the fluid flow manifold including a channel having straight portion and an annular portion, and the second side of the fluid flow manifold having a cavity, the cavity including a first aperture for allowing fluid flow in a first direction, and one or more further apertures for allowing fluid flow in a second direction; a sonication chamber including the cavity of the fluid flow manifold; an ultrasonic transducer positioned between the sonication chamber and the second end wall, the ultrasonic transducer having an atomisation surface adjacent to the sonication chamber and in communication with the sonication chamber; a transducer holder couplable to at least one of the spacer and the fluid flow manifold, and including a lower disc portion, an upper annular portion, and a gasket positioned therebetween, an air inlet conduit for conducting air to the sonication chamber; and a mist outlet conduit for conducting mist to the mist outlet port, wherein, in use, the liquid is conducted from the liquid chamber to the sonication chamber to be atomised, and the mist generated is conducted through the mist outlet port.

In some examples, there is provided a mist inhalation pod for use with a driver, the pod comprising: a housing having a first end, an opposite second end and at least one side wall extending between the first end and the second end; a first end wall proximate the first end and the side wall, the first end wall closing the first end of the housing, the first end wall being provided with a mist outlet port; a second end wall proximate the second end and the side wall, the second end wall closing the second end of the housing; a liquid barrier wall positioned within the housing and spaced apart from the first end wall, the liquid barrier wall having at least one liquid channel with a liquid inlet and a liquid outlet; a liquid chamber partially defined by the liquid barrier wall, the liquid chamber containing a liquid to be atomised, the liquid comprising a therapeutic, the liquid inlet being in liquid communication with the liquid chamber for conducting the liquid through the liquid outlet; a spacer positioned within the housing between the liquid barrier wall and the second end wall, the spacer including a hollow interior surrounded by a perimeter; a fluid flow manifold positioned at least partially within the hollow interior of the spacer and including a first side proximate the first end wall, and a second side proximate the second end wall, the first side of the fluid flow manifold including a channel having straight portion and an annular portion, and the second side of the fluid flow manifold having a cavity, the cavity including a first aperture for allowing fluid flow in a first direction, and one or more further apertures for allowing fluid flow in a second direction; a sonication chamber including the cavity of the fluid flow manifold; an ultrasonic transducer positioned between the sonication chamber and the second end wall, the ultrasonic transducer having an atomisation surface adjacent to the sonication chamber and in communication with the sonication chamber; a transducer holder configured to retain the ultrasonic transducer, the transducer holder being couplable to at least one of the spacer and the fluid flow manifold, the transducer holder including an upper annular portion and an annular retaining ring, the annular retaining ring positioned between the upper annular portion and the ultrasonic transducer; an air inlet conduit for conducting air to the sonication chamber; and a mist outlet conduit for conducting mist to the mist outlet port, wherein, in use, the liquid is conducted from the liquid chamber to the sonication chamber to be atomised, and the mist generated is conducted through the mist outlet port.

In some examples, there is provided a capillary for use with a mist inhalation pod, the capillary comprising: a first portion configured to be at least partly superimposed on an atomisation surface of an ultrasonic transducer, the first portion being partly circular in shape; and a second portion configured to conduct a liquid to be atomised to the first portion, the second portion having a generally rectangular shape, and the liquid comprising a therapeutic, the first portion including a first surface defining a first side of the capillary and a second surface defining a second side of the capillary, the first surface configured to be in contact with the atomisation surface of the ultrasonic transducer, the first portion including an opening which enables bubbles trapped between the capillary and the atomisation surface of the ultrasonic transducer to pass from the first side of the capillary to the second side of the capillary.

In some examples, the opening is a through hole extending from the first side of the capillary to the second side of the capillary.

In some examples, the through hole is positioned in the centre of the first portion of the capillary.

In some examples, a plurality of through holes are provided in the first portion of the capillary.

In some examples, the plurality of through holes are spaced around the centre of the first portion of the capillary.

In some examples, the opening is a slit in the first portion of the capillary.

In some examples, the slit passes through the centre of the first portion of the capillary.

In some examples, the first portion of the capillary includes a plurality of slits.

In some examples, the plurality of slits join together at the centre of the first portion of the capillary.

In some examples, there is provided a mist inhalation pod for use with a driver, the pod comprising: a housing; a liquid chamber within the housing, the liquid chamber containing a liquid to be atomised, the liquid comprising a therapeutic, a sonication chamber; an ultrasonic transducer having an atomisation surface adjacent to the sonication chamber and in communication with the sonication chamber; a capillary having a first portion at least partly superimposed on the atomisation surface of the ultrasonic transducer and a second portion in communication with the liquid chamber, the first portion being partly circular in shape and the second portion being generally rectangular in shape, the second portion of the capillary configured to conduct the liquid from the liquid chamber to the first portion of the capillary, the first portion including a first surface defining a first side of the capillary and a second surface defining a second side of the capillary, the first portion including an opening which enables bubbles trapped between the capillary and the atomisation surface to pass from the first side of the capillary to the second side of the capillary.

In some examples, the opening is a through hole extending from the first side of the capillary to the second side of the capillary.

In some examples, the through hole is positioned in the centre of the first portion of the capillary.

In some examples, a plurality of through holes are provided in the first portion of the capillary.

In some examples, the plurality of through holes are spaced around the centre of the first portion of the capillary.

In some examples, the opening is a slit in the first portion of the capillary.

In some examples, the slit passes through the centre of the first portion of the capillary.

In some examples, the first portion of the capillary includes a plurality of slits.

In some examples, the plurality of slits join together at the centre of the first portion of the capillary.

In some examples, there is provided a mist inhalation device comprising a pod and a driver, the pod comprising: a housing; a liquid chamber within the housing, the liquid chamber containing a liquid to be atomised, the liquid comprising a therapeutic, a sonication chamber; an ultrasonic transducer having an atomisation surface adjacent to the sonication chamber and in communication with the sonication chamber; a capillary having a first portion at least partly superimposed on the atomisation surface of the ultrasonic transducer and a second portion in communication with the liquid chamber, the first portion being partly circular in shape and the second portion being generally rectangular in shape, the second portion of the capillary configured to conduct the liquid from the liquid chamber to the first portion of the capillary, the first portion including a first surface defining a first side of the capillary and a second surface defining a second side of the capillary, the first portion including an opening which enables bubbles trapped between the capillary and the atomisation surface to pass from the first side of the capillary to the second side of the capillary, the driver including: a housing having a cavity configured to receive at least part of the pod; and driver circuitry for supplying a drive signal to the pod.

In some examples, the driver housing includes a hole, and the driver includes a conduit extending from the hole to the pod, the conduit configured to convey air from the surroundings to the air inlet conduit of the pod.

In some examples, there is provided a capillary for use with a mist inhalation pod, the capillary comprising: a first portion configured to be at least partly superimposed on an atomisation surface of an ultrasonic transducer, the first portion having at least one arcuate edge, and a second portion configured to conduct a liquid comprising a therapeutic to the first portion, the second portion having a generally rectangular shape including two substantially parallel edges, the arcuate edge of the first portion extending from one of the substantially parallel edges of the two substantially parallel edges of the second portion.

In some examples, the capillary further comprises: a third portion configured to conduct the liquid to the first portion, the third portion having a generally rectangular shape including two substantially parallel edges, the third portion being positioned on an opposing side of the first portion to the second portion, the arcuate edge of the first portion extending from the one of the two substantially parallel edges of the second portion to one of the two substantially parallel edges of the third portion.

### BRIEF DESCRIPTION OF THE FIGURES

In order that the present disclosure may be more readily understood, preferable embodiments thereof will now be described, by way of example only, with reference to the accompanying drawings, in which:

FIG. 1 is a diagrammatic view of a mist inhalation pod embodying the present disclosure;

FIG. 2 is a diagrammatic cross-sectional view of the mist inhalation pod, embodying the present disclosure;

FIG. 3 is a diagrammatic cross-sectional view of a part of the mist inhalation pod, embodying the present disclosure;

FIG. 4 is a diagrammatic exploded view of a part of the mist inhalation pod, embodying the present disclosure;

FIG. 5 is a diagrammatic view of a part of the mist inhalation pod, embodying the present disclosure;

FIG. 6 is a diagrammatic view of a part of the mist inhalation pod, embodying the present disclosure;

FIG. 7 is a diagrammatic view of the part of the mist inhalation pod illustrated in FIG. 6;

FIG. 8 is a diagrammatic view of a part of the mist inhalation pod, embodying the present disclosure;

FIG. 9 is a diagrammatic view of the mist inhalation pod, embodying the present disclosure;

FIG. 10 is a diagrammatic view of a part of the mist inhalation pod, embodying the present disclosure;

FIG. 11 is a diagrammatic cross-sectional view of the part of the mist inhalation pod illustrated in FIG. 10, embodying the present disclosure;

FIG. 12 is a diagrammatic cross-sectional view of a part of the mist inhalation pod, embodying the present disclosure;

FIG. 13 is a diagrammatic view of a mist inhalation pod embodying the present disclosure;

FIG. 14 is a diagrammatic cross-sectional view of the mist inhalation pod, embodying the present disclosure;

FIG. 15 is a diagrammatic cross-sectional view of a part of the mist inhalation pod, embodying the present disclosure;

FIG. 16 is a diagrammatic exploded view of a part of the mist inhalation pod, embodying the present disclosure;

FIG. 17 is a diagrammatic view of a part of the mist inhalation pod, embodying the present disclosure;

FIG. 18 is a diagrammatic view of a part of the mist inhalation pod, embodying the present disclosure;

FIG. 19 is a diagrammatic view of the part of the mist inhalation pod illustrated in FIG. 18;

FIG. 20 is a diagrammatic view of a part of the mist inhalation pod, embodying the present disclosure;

FIG. 21 is a diagrammatic view of the mist inhalation pod, embodying the present disclosure;

FIG. 22 is a diagrammatic view of a part of the mist inhalation pod, embodying the present disclosure;

FIG. 23 is a diagrammatic cross-sectional view of the part of the mist inhalation pod illustrated in FIG. 22, embodying the present disclosure;

FIG. 24 is a diagrammatic cross-sectional view of a part of the mist inhalation pod, embodying the present disclosure;

FIG. 25 is a diagrammatic view of a capillary for use with a mist inhalation pod in a first configuration, embodying the present disclosure;

FIG. 26 is a diagrammatic view of the capillary as in FIG. 25 in a second configuration;

FIG. 27 is a diagrammatic view of a capillary for use with a mist inhalation pod in a first configuration, embodying the present disclosure;

FIG. 28 is a diagrammatic view of the capillary as in FIG. 27 in a second configuration;

FIG. 29 is a diagrammatic view of a capillary for use with a mist inhalation pod in a first configuration, embodying the present disclosure;

FIG. 30 is a diagrammatic view of the capillary as in FIG. 29 in a second configuration;

FIG. 31 is a diagrammatic view of a capillary for use with a mist inhalation pod in a first configuration, embodying the present disclosure;

FIG. 32 is a diagrammatic view of the capillary as in FIG. 31 in a second configuration;

FIG. 33 is a diagrammatic view of a capillary for use with a mist inhalation pod in a first configuration, embodying the present disclosure; and

FIG. 34 is a diagrammatic view of the capillary as in FIG. 33 in a second configuration.

### DETAILED DESCRIPTION OF THE DISCLOSURE

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

The following disclosure provides many different embodiments, or examples, for implementing different features of the subject matter provided. Specific examples of components, concentrations, applications and arrangements are described below to simplify the present disclosure. These are, of course, merely examples and are not intended to be limiting. For example, the attachment of a first feature and a second feature in the description that follows may include embodiments in which the first feature and the second feature are attached in direct contact, and may also include embodiments in which additional features may be positioned between the first feature and the second feature, such that the first feature and the second feature may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The following disclosure describes representative arrangements or examples. Each arrangement or example may be considered to be an embodiment and any reference to an “arrangement” or an “example” may be changed to “embodiment” in the present disclosure.

Although the figures and description may indicate that there are two primary embodiments, each of the components of each embodiment are interchangeable where technically possible as it will be understood to be impractical to list or illustrate every possible permutation of components.

Conventional electronic vaporizing inhalers tend to rely on inducing high temperatures of a metal component configured to heat a liquid in the inhaler, thus vaporizing the liquid that can be breathed in. The liquid typically contains nicotine and flavorings blended into a solution of propylene glycol (PG) and vegetable glycerin (VG), which is vaporized via a heating component at high temperatures. Problems with conventional inhalers may include the possibility of burning metal and subsequent breathing in of the metal along with the burnt liquid. In addition, some may not prefer the burnt smell or taste caused by the heated liquid.

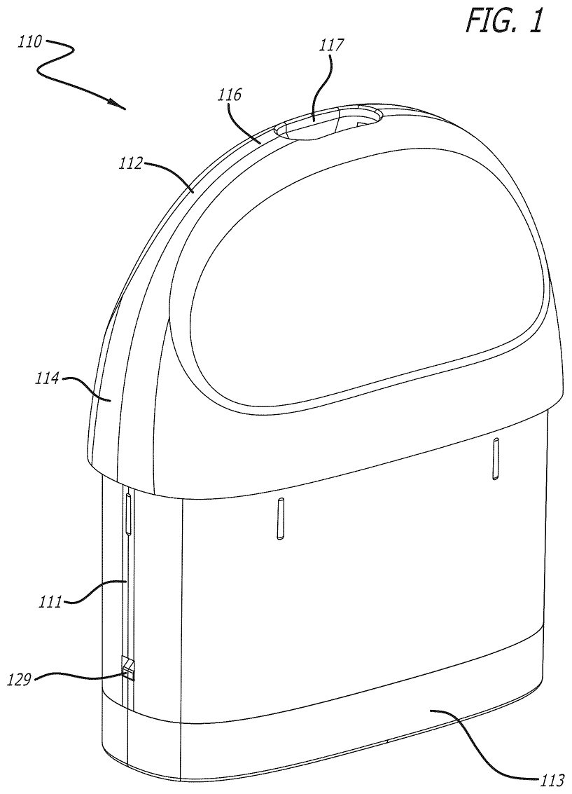

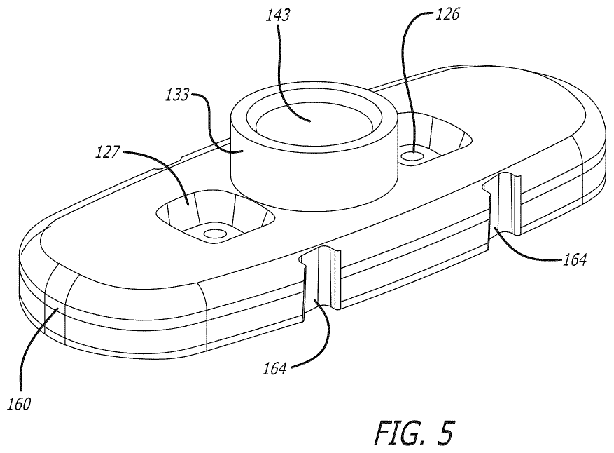

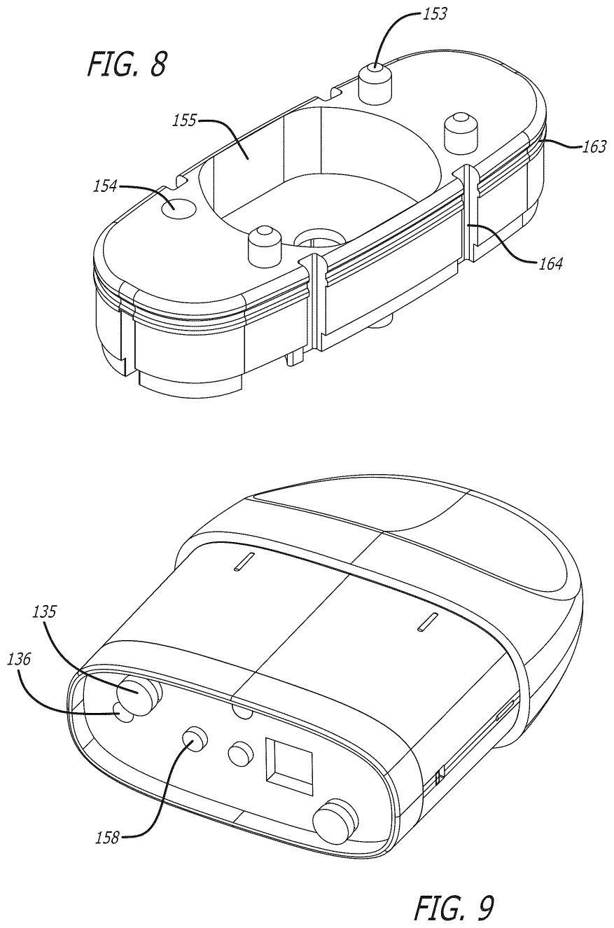

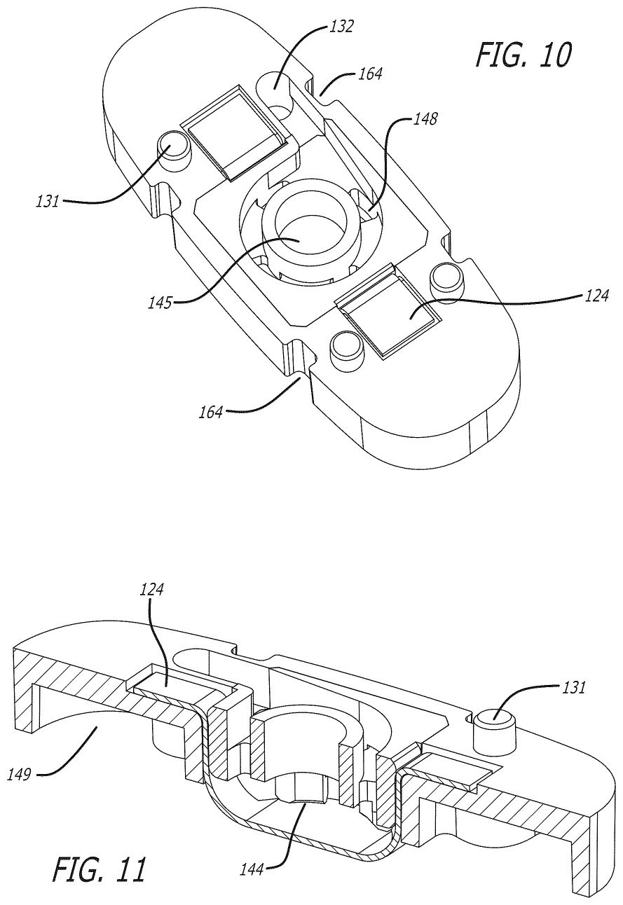

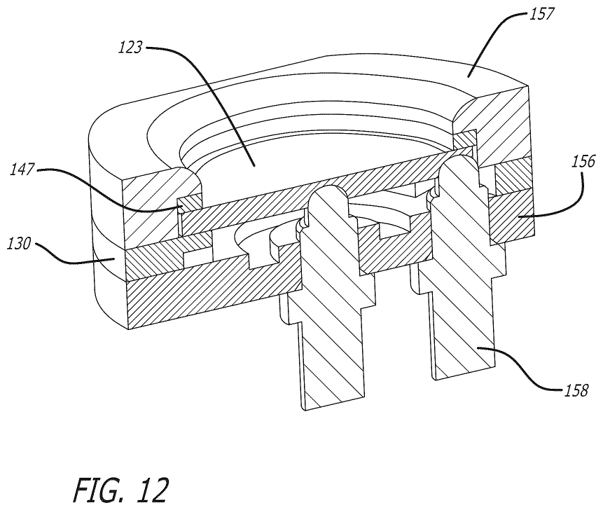

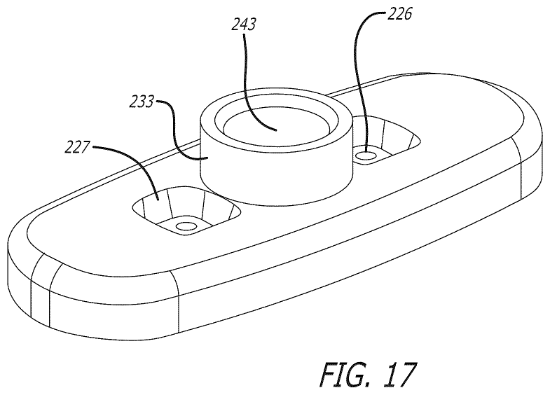

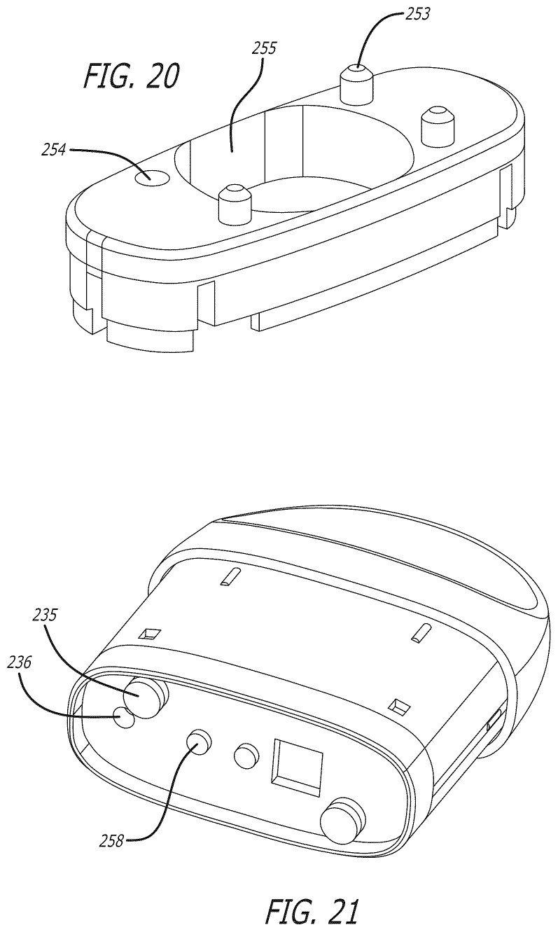

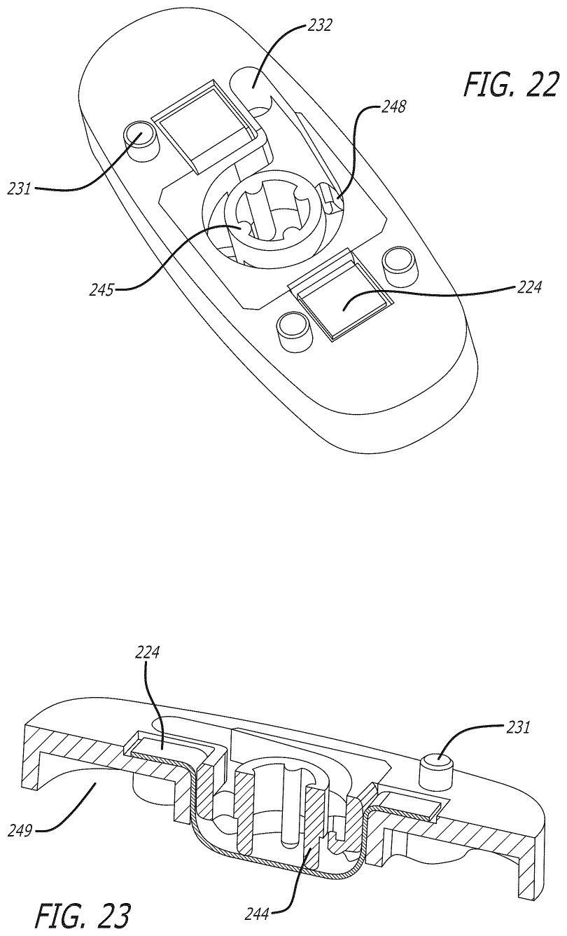

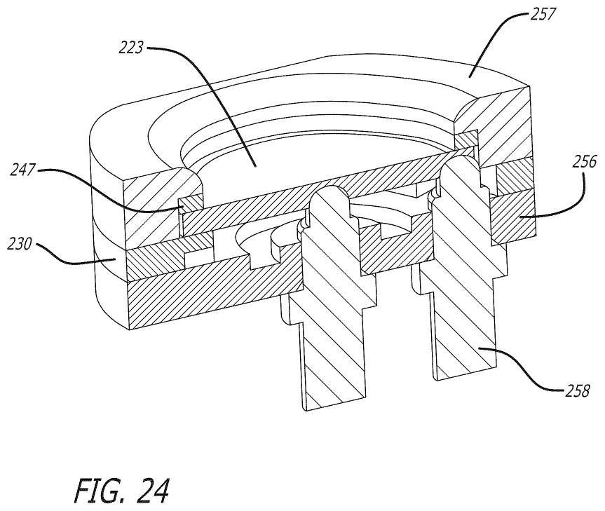

FIGS. 1 to 24 illustrate a pod 110, 210, or components thereof, comprising a sonication chamber. It is noted that the expression “mist” used in the following disclosure means the liquid is not heated as in traditional inhalers known from the prior art. In fact, traditional inhalers use heating elements to heat the liquid above its boiling temperature to produce a vapor, which is different from a mist. A vapor involves a phase change from the liquid to a gas, whereas the liquid dispersed within the air in the present disclosure remains in the liquid phase.

When sonicating liquids at high intensities, the sound waves that propagate into the liquid media result in alternating high-pressure (compression) and low-pressure (rarefaction) cycles, at different rates depending on the frequency. During the low-pressure cycle, high-intensity ultrasonic waves create small vacuum bubbles or voids in the liquid. This phenomenon is termed cavitation. When the bubbles attain a volume at which they can no longer absorb energy, they collapse violently during a high-pressure cycle. During the implosion, very high pressures are reached locally. At cavitation, broken capillary waves are generated, and tiny droplets break the surface tension of the liquid and are quickly released into the air, taking mist form.

The following will explain more precisely the cavitation phenomenon.

When the liquid is atomized by ultrasonic vibrations, micro water bubbles are produced in the liquid.

The bubble production is a process of formation of cavities created by the negative pressure generated by intense ultrasonic waves generated by the means of ultrasonic vibrations.

High intensity ultrasonic sound waves leading to rapid growth of cavities with relatively low and negligible reduction in cavity size during the positive pressure cycle.

Ultrasound waves, like all sound waves, consist of cycles of compression and expansion. When in contact with a liquid, compression cycles exert a positive pressure on the liquid, pushing the molecules together. Expansion cycles exert a negative pressure, pulling the molecules away from one another.

Intense ultrasound waves create regions of positive pressure and negative pressure. A cavity can form and grow during the episodes of negative pressure. When the cavity attains a critical size, the cavity implodes.

The amount of negative pressure needed depends on the type and purity of the liquid. For truly pure liquids, tensile strengths are so great that available ultrasound generators cannot produce enough negative pressure to make cavities. In pure water, for instance, more than 1,000 atmospheres of negative pressure would be required, yet the most powerful ultrasound generators produce only about 50 atmospheres of negative pressure. The tensile strength of liquids is reduced by the gas trapped within the crevices of the liquid particles. The effect is analogous to the reduction in strength that occurs from cracks in solid materials. When a crevice filled with gas is exposed to a negative-pressure cycle from a sound wave, the reduced pressure makes the gas in the crevice expand until a small bubble is released into solution.

However, a bubble irradiated with ultrasound continually absorbs energy from alternating compression and expansion cycles of the sound wave. These cause the bubbles to grow and contract, striking a dynamic balance between the void inside the bubble and the liquid outside. In some cases, ultrasonic waves will sustain a bubble that simply oscillates in size. In other cases, the average size of the bubble will increase.

Cavity growth depends on the intensity of sound. High-intensity ultrasound can expand the cavity so rapidly during the negative-pressure cycle that the cavity never has a chance to shrink during the positive-pressure cycle. In this process, cavities can grow rapidly in the course of a single cycle of sound.

For low-intensity ultrasound the size of the cavity oscillates in phase with the expansion and compression cycles. The surface of a cavity produced by low-intensity ultrasound is slightly greater during expansion cycles than during compression cycles. Since the amount of gas that diffuses in or out of the cavity depends on the surface area, diffusion into the cavity during expansion cycles will be slightly greater than diffusion out during compression cycles. For each cycle of sound, then, the cavity expands a little more than it shrinks. Over many cycles the cavities will grow slowly.

It has been noticed that the growing cavity can eventually reach a critical size where it will most efficiently absorb energy from the ultrasound. The critical size depends on the frequency of the ultrasound wave. Once a cavity has experienced a very rapid growth caused by high intensity ultrasound, it can no longer absorb energy as efficiently from the sound waves. Without this energy input the cavity can no longer sustain itself. The liquid rushes in and the cavity implodes due to a non-linear response.

The energy released from the implosion causes the liquid to be fragmented into microscopic particles which are dispersed into the air as mist.

The following description is generally applicable to embodiments shown in both FIGS. 1 to 12 and 13 to 24, unless specified otherwise. As mentioned above, however, this does not prevent a feature and/or component from being used in other embodiments or examples. Similarly, the described capillary may be used with any pod, whether that pod is described or not.

FIGS. 1 and 13 show a mist inhalation pod (hereinafter referred to as a pod) 110, 210 according to some embodiments if the present disclosure. The pod 110, 210 is configured to be releasably attached to a driver (not shown). The driver houses the components required to store electrical energy and provide an electrical signal to the pod 110, 210. In other examples, the pod 110, 210 may be fixed to, formed integrally with or otherwise non-releasably attached to the driver.

The pod 110, 210 comprises a housing 111, 211 having a first end and an opposite second end, a mouthpiece 112, 212 and an end cap 113, 213. Between the two ends of the housing 111, 211 extends at least one side wall. In some examples, the housing 111, 211 is of injection moulded plastic, specifically polypropylene that is typically used for medical applications. In some examples, the housing 111, 211 is of a heterophasic copolymer. More particularly a BF970MO heterophasic copolymer is preferred, which has an optimum combination of very high stiffness and high impact strength. Parts moulded with this material also exhibit good anti-static performance.

A heterophasic copolymer such as polypropylene is particularly suitable for the pod housing 111, 211 since this material minimises or does not cause condensation of the aerosol as it flows through the mouthpiece 112, 212 to the user. This plastic material can also be directly recycled easily using industrial shredding and cleaning processes.

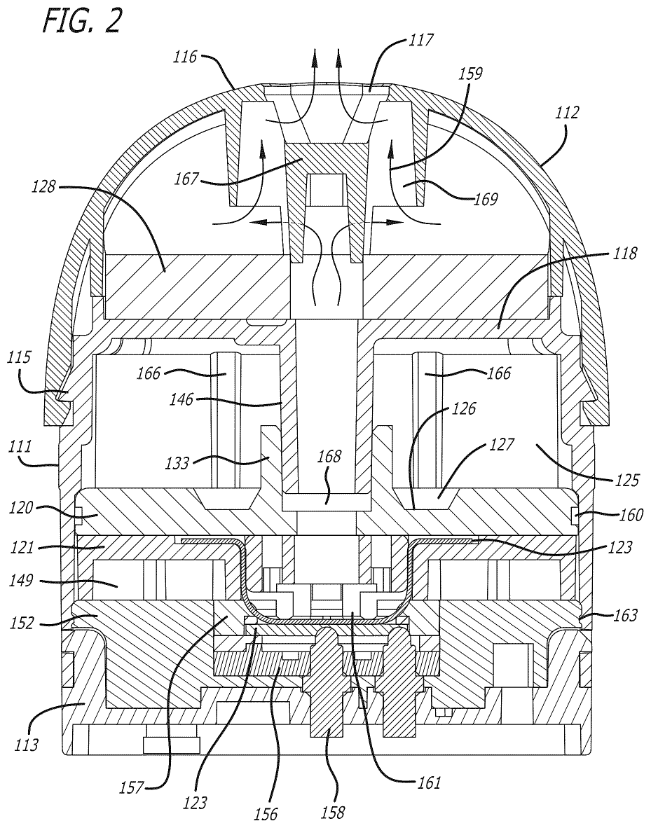

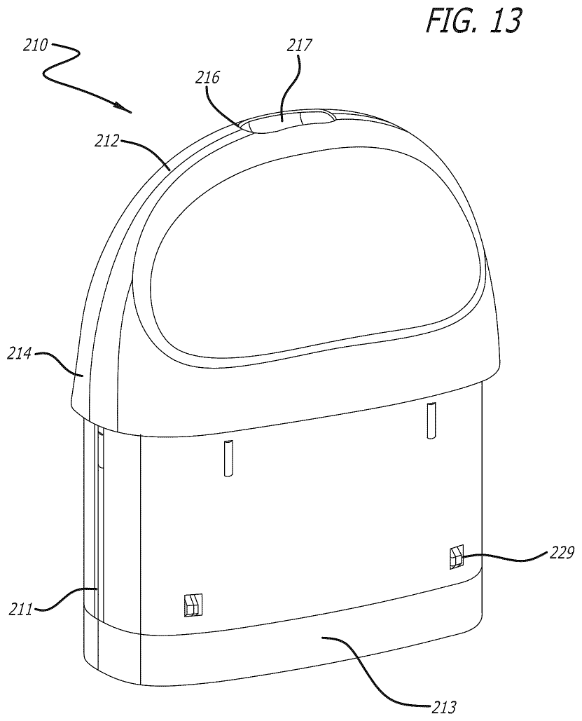

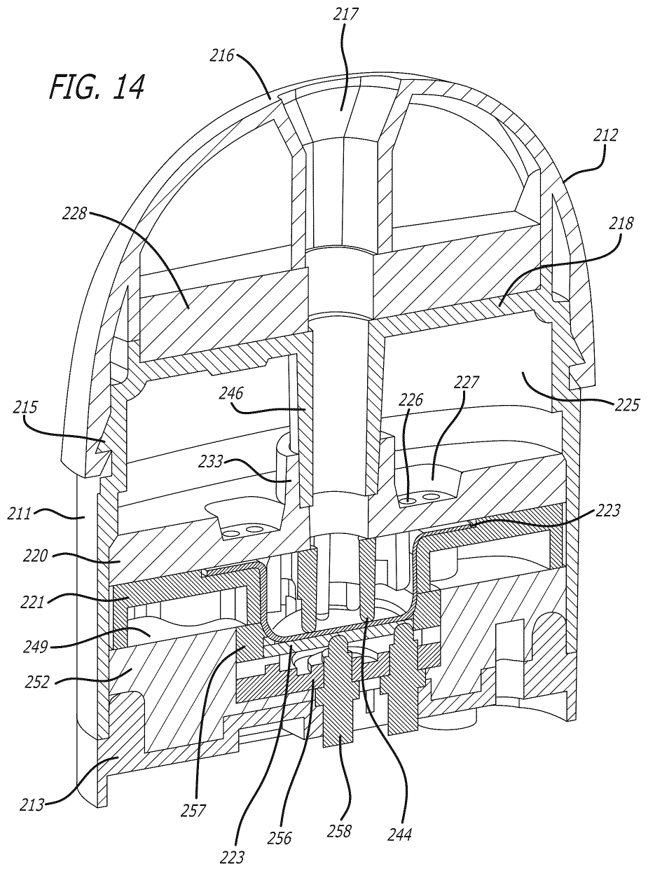

The mouthpiece 112, 212 comprises a base 114, 214 having an opening which receives a connector portion 115, 215 positioned towards the first end of the housing 111, 211, as seen in FIGS. 2 and 14. The connector portion 115, 215 may comprises at least one latch element that engages a latch recess to retain the mouthpiece 112, 212 in connection with the housing 111, 211.

The mouthpiece 112, 212 may narrow progressively from the base 114, 214 to a distal end 116, 216. The distal end 116, 216 comprises a mouthpiece outlet port 117, 217 to enable mist to exit the pod 110, 210 for inhalation by the user. The mouthpiece 112, 212 may be substantially oval in cross section to allow for comfortable use by the user. The longer sides of the mouthpiece 112, 212 may be indented in order to further increase the user comfort and experience.

The mouthpiece 112, as shown in FIG. 2, may comprise an indirect flow path for mist produced by the pod 110 so that the mist takes a non-direct path to the mouthpiece outlet port 117. The mist is forced to take a path which is diverted radially outward and then radially inward again before passing through the mouthpiece outlet port 117. The mist flow path may further include a tangential component, or any flow path which increases the dwell time of the mist within the mouthpiece 112. One example of such a mist flow path is illustrated by the arrows 159 in FIG. 2. It will be understood that a non-direct flow path includes any flow path which includes a deviation from a straight line through the mouthpiece 112. A non-direct flow path reduces the likelihood of liquid droplets being drawn through the mouthpiece by the user, and increasing the dwell time allows for more liquid droplets to separate from the mist flow.

The mist preferably flows into the mouthpiece 112 through a mouthpiece inlet port which is in communication with a mist generating component. The mouthpiece 112 may act as a chamber through which the mist passes on its way to the mouthpiece outlet port 117. The mouthpiece chamber is of a larger diameter, and preferably of a much larger diameter, than the mist outlet conduit 146 and the mouthpiece outlet port 117.

A blocking element 167 may be positioned axially between the mouthpiece inlet port and the mouthpiece outlet port 117 to prevent the mist exiting the pod 110 straight through the mouthpiece outlet port 117 without first entering the mouthpiece chamber. The blocking element 167 may, as shown in FIG. 2, have a substantially horizontal plate portion which serves to interrupt the mist flow path. In some examples, the plate portion may not be horizontal and may instead be angled to deflect the mist flow in at least one specific direction. The blocking element 167 may further include at least one, but preferably a plurality of legs which extend longitudinally with respect to the pod 110. The legs may extend as far as an absorbing element 128 or the mist outlet conduit 146 to aid assembly and to prevent mist from bypassing the blocking element 167.

The blocking element 167 may further include at least one mist outlet aperture 169. The mist outlet aperture 169 is in fluid communication with both the mouthpiece chamber and the mouthpiece outlet port 117. The mouthpiece outlet aperture(s) 169 are preferably positioned at approximately 90 degrees to the mist flow path into the mouthpiece chamber, so as to increase the dwell time of the mist in the mouthpiece chamber. In other words, the legs of the blocking element 167 preferably block a direct path between the mouthpiece inlet port and the mouthpiece outlet aperture 169.

Although the blocking element 167 is described as a component, it could instead be integrated into the mouthpiece 112.

The end cap 113, 213 attaches to the second end of the housing 111, 211. The end cap 113, 213 includes a plurality of deformable hooked elements 129, 229 for receiving and securing the side wall of the housing 111, 211. The underside of the end cap 113, 213 includes a recess, most clearly shown in FIGS. 9 and 21, for housing circuitry.

FIGS. 2 and 14 illustrate the internal components of the pod 110, 210 according to some examples. The housing 111, 211 comprises a first end wall 118, 218 proximate the first end of the housing 111, 211, and a second end wall in the form of the end cap 113, 213. The first end wall 118, 218 and the second end wall may be configured to close the first end and the second end of the housing 111, 211, respectively. The side wall of the housing 111 may comprise at least one, but preferably a plurality of ridges 166, as shown in FIG. 2. The ridges 166 may be configured to engage corresponding longitudinal grooves 164 of the liquid barrier wall 120, the spacer 121, and the lower body portion 152. The ridge 166 and groove 164 interaction eases the assembly of the pod 110, where it is important that the internal components of the pod 110 align correctly with one another. If misalignment occurs, the air inlet conduit may not be correctly formed, thereby causing inefficiencies in the assembled pod 110. In some examples, at least one of the ridges 166 may be of differing size and/or shape to the others so that the pod 110 can only be assembled correctly without inadvertently placing a component incorrectly.

An absorbing element 128, 228 may be positioned in or adjacent to the mist flow path so as to absorb any liquid droplets as the mist is conducted towards the mouthpiece 112, 212. Preferably, the absorbing element 128, 228 is at least partly of bamboo fibre. In some examples, the absorbing element 128, 228 is positioned within the mouthpiece 112, 212. The absorbing element 128, 228 preferably extends radially to the wall of the mouthpiece 112, 212 to increase the volume of mist droplets absorbed.

The pod 110, 210 comprises a mist outlet conduit. In some examples, at least a first section 46 of the mist outlet conduit may be integrally formed with the housing 111, 211 and extend from the first end wall 118, 218 towards the second end wall.

The housing 111, 211 may enclose a liquid barrier wall 120, 220, a spacer 121, 221, a fluid flow manifold 122, 222, an ultrasonic transducer 123, 223, and a capillary 124, 224.

The liquid barrier wall 120, 220 is positioned within the housing 111, 211 and extends towards the side wall to create a seal between the liquid barrier wall 120, 220 and the side wall of the housing 111, 211. In some examples, the liquid barrier wall 120 has a contoured outer edge such that the outer edge contacts the side wall in two distinct locations, thereby providing a double seal against the side wall. The double seal may be provided by a circumferential groove 160. The liquid barrier wall 120, 220 is spaced apart from the first end wall 118, 218 to form a liquid chamber 125, 225 therebetween. The liquid chamber 125, 225 is configured to hold a liquid to be atomised.

The liquid chamber 125, 225 is configured to hold a liquid to be atomised. The liquid preferably includes at least one therapeutic suitable for the aerosol delivery to the lungs through inhalation by a patient for providing a desired treatment to the patient. Some examples of therapeutics include, but are not limited to, aerosol delivery of pharmacological agents to the lungs to promote a systemic or direct clinical effect while producing a minimal adverse-effect. A therapeutic may also include for example, but not limited to, natural drugs, cannabinoid derivatives such as CBD for pain relief and other treatments; botanicals; opioids; RNA; DNA; chemotherapies; subcellular components including for example ribosomes, endoplasmic reticulum, cytoskeleton, and mitochondria; supplements for performance enhancement; albuterol/salbutamol for asthma patients; agents such as beta-lactams, polymyxins, and aminoglycosides bactericidal antibiotics; amphotericin B; morphine; fentanyl; prostacyclin, amiloride, and interferon-g; and cyclosporine as rescue therapy rejection in lung-transplantation patients and a treatment for asthma.

In some examples, the liquid chamber 125, 225 contains an e-liquid or liquid comprising at least one cannabinoid or phytocannabinoid derived from a cannabis plant. In some examples, the at least one cannabinoid or phytocannabinoid comprises one or more of tetrahydrocannabinol (THC), cannabidiol (CBD) and/or cannabinol (CBN).

In some examples, the liquid chamber 125, 225 contains an e-liquid or liquid comprising a psychedelic compound or compounds for use as a therapeutic. In some examples, the psychedelic compound or compounds is one or more of lysergic acid diethylamide (LSD), 3,4-methylenedioxymethamphetamine (MDMA), ketamine, esketamine, ibogaine, mescaline, a tryptamine, a substituted tryptamine, O-acetylpsilocin (4-AcO-DMT), psilocybin (4-PO-DMT), psilocin (4-HO-DMT), O-methylbufotenin (5-MeO-DMT), ufotenine (5-HO-DMT) and/or N,N-Dimethyltryptamine (DMT or N, N-DMT).

The devices disclosed herein are for use with any therapeutics, drugs or other compounds, with the drug or compound being provided in a liquid within the liquid chamber of the device for aerosolisation by the device. In some examples, the devices disclosed herein are for use with therapeutics, drugs and compounds including, but not limited to, the following:
Respiratory
Brochodilators
Olodaterol
Levalbuterol
Berodual (Ipratropium bromide/Fenoterol)
Combivent (Ipratropium bromide/Salbutamol)
Anti-inflammatory
Betamethasone
Dexamethasone
Methylprednisolone
Hydrocortisone
Mucolytics
N-Acetylcysteine
Pulmonary Hypertension
Sildenafil
Tadalafil
Epoprostenol
Treprostenil
Iloprost
Infectious Disease
Antimicrobials
Aminoglycosides (Gentamicin, Tobramycin, Amikacin, Colomycin, Neomycin, Liposomal Amikacin,)
Quinolones (Ciprofloxacin, Levofloxacin, Moxifloxacin Ofloxacin)
Macrolides (Azithromycin)
Minocycline
Betalactams (Piperacillin-Tazobactam, Ceftazidime Ticarcillin)
Cephalosporins (Cefotaxime, Cefepime, Ceftriaxone, Cefotaxime)
Glycopeptides (Vancomycin)
Meropenem
Polymixin (Colistin, Polymixin B)
Antifungals
Amphotericin
Fluconazole
Caspofungen
Antivirals
Valganciclovir
Favipiravir
Remdisivir
Acyclovir
Anti TB
Isoniazid
Pyrazinamide
Rifampin
Ethambutol
Oncology
Biologics
Gilotrif
Afatinib
Caplacizumab
Dupilumab
Isarilumab
Alirucomab_
Volasertib_
Nintedanib
Imatinib
Sirolimus
Chemotherapy
Azacitidine
Decitabine
Docetaxel
Gemcitabine
Cisplatinum
CNS & PSYCH
Sodium valproate
Teriflunomide
Zomitriptan
METABOLIC/HORMONAL
Insulin
Estrogen
IMMUNOLOGY
Vaccine
Monoclonal Antibodies
Stem Cells
Vitamins
Zinc
Ascorbic Acid
Miscellaneous
Niclosamide
Hydroxychloroquine
Ivermectin

In some examples, the liquid chamber has a volume of approximately 2.5 ml. In other examples, this may be reduced or increased, such as to comply with legal requirements or determined user preferences and requirements.

The liquid barrier wall 120, 220 comprises a liquid channel 126, 226 having a liquid inlet and a liquid outlet. The liquid channel 126, 226 passes entirely through the liquid barrier wall 120, 220 so as to allow liquid communication between the liquid chamber 125, 225 and the capillary 124, 224. The liquid barrier wall 120, 220 may include more than one liquid channel 126, 226 to improve the liquid flow rate to the capillary 124, 224, or to improve the dispersion of the liquid over a larger surface area of the capillary 124, 224. The diameter and the number of the liquid channels 126, 226 are chosen to allow sufficient liquid flow to the capillary 124, 224 without allowing a significant oversupply to the capillary 124, 224, and as such prevent flooding and leaking, and also allows for more efficient mist generation.

The liquid barrier wall 120, 220 may further comprise one or more recesses 127, 227 in the planar face that defines the liquid chamber 125, 225. Providing the liquid inlet of the liquid channel 126, 226 in the recess 127, 227 allows the liquid chamber 125, 225 to be fully depleted of liquid before the pod 110, 210 needs to be refilled or disposed of, due to the recess 127, 227 representing the lowest point in the liquid chamber 125, 225. As illustrated in FIGS. 2 and 14, the recesses 127, 227 and liquid channels may be positioned as close as possible to the sonication chamber 142, 242 radially, thereby reducing the length of capillary 124, 224 required and thus saving materials and manufacturing costs. Further, the recesses preferably span less than 50%, and more preferably less than 40% of the distance between the central protrusion 133, 233 and the furthest point of the liquid barrier wall 120, 220 from the central protrusion 133, 233. Such dimensions may optimise the drainage of the liquid chamber 125, 225.

The liquid barrier wall 120, 220 may comprise blind holes in its lower surface (i.e. the planar surface opposite that on which the recess 127, 227 is provided) configured to accept pegs 131, 231 of the spacer 121, 221.

The liquid barrier wall 120, 220 may further comprise a central protrusion 133, 233 which includes a through hole 143, 243. The central protrusion 133, 233 and through hole 143, 243 form a second section of the mist outlet conduit.

In some examples, a mist diverter assembly (not shown) may be positioned within the bore of the through hole. The purpose of the mist diverter assembly is to prevent, or at least reduce the number of, large liquid droplets from flowing to the mouthpiece 112, 212 and, ultimately, into the mouth of the user. The mist diverter assembly may comprise a plate, which may be supported by a plurality of splines spaced around the bore of the through hole. In examples including a mist diverter assembly, the mist diverter assembly may be provided instead of or in addition to the mist diversion configuration provided in the mouthpiece 112.

The spacer 121, 221 is positioned between the liquid barrier wall 120, 220 and the second end wall within the housing 111, 211. The spacer 121, 221 has an outer wall forming a perimeter, and a hollow interior. Once the pod 110, 210 is assembled, the perimeter of the spacer 121, 221 extends towards the side wall of the housing 111, 211. In order to save weight and material costs, the underside of the spacer, i.e., the side proximate the second end wall, may include at least one cavity 149, 249.

The spacer 121, 221 includes a slot 132, 232 which may extend axially through the perimeter portion. In some examples, the slot 132, 232 may not extend through the top surface of the perimeter portion, the top surface being the surface which may abut the liquid barrier wall 120, 220. The slot 132, 232 forms a section of the air inlet conduit once the pod 110, 210 is assembled.

The spacer 121, 221 further includes indentations 137, 237 for accepting at least part of the second portion of the capillary 124, 224. The indentations 137, 237 may align with the liquid channels 126, 226 of the liquid barrier wall 120, 220 and thereby permit at least part of the second portion of the capillary 124, 224 to lie adjacent the liquid outlet of the liquid channels 126, 226. It will be appreciated that the spacer 121, 221 may include only one indentation 137, 237, or need not include indentations 137, 237 at all.

In some envisaged alternative examples, the spacer 121, 221 may be spaced from the lower surface of the liquid barrier wall 120, 220. In a further alternative, indentations may additionally or alternatively be provided in the lower surface of the liquid barrier wall 120, 220.

At least a part of at least one of the liquid barrier wall 120, 220, the spacer 121, 221, and the second end wall may be at least partly of a resiliently deformable material so as to prevent liquid leaking from the liquid chamber 125, 225. Such a material may comprise silicone.

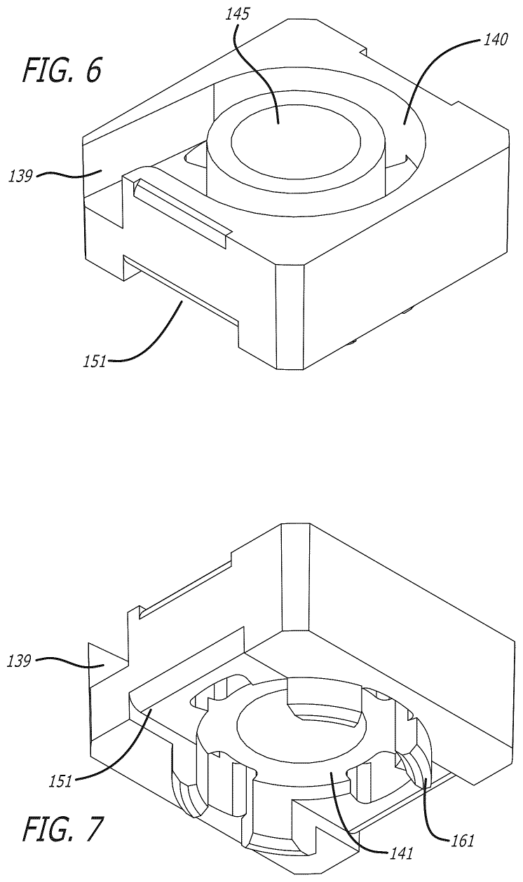

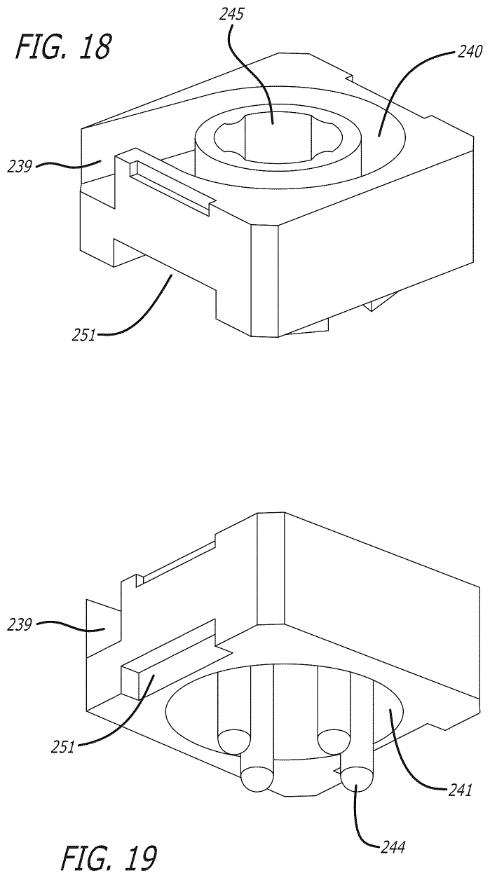

The hollow interior of the spacer 121, 221 is sized to at least partially receive the fluid flow manifold 122, 222. The manifold 122, 222 has a first side and an opposite second side, the first side being the upper side as shown in FIGS. 6 and 18, and the second side being the lower side shown in FIGS. 7 and 19. When assembled, the first side of the manifold 122, 222 is proximate the first end wall 118, 218 and the second side of the manifold is proximate the second end wall.

The first side of the manifold 122, 222 may comprise a channel 138, 238. The channel 138, 238 extends from an edge of the manifold 122, 222 which is proximate the slot 132, 232 in the spacer 121, 221. The channel 138, 238 may comprise a first portion 139, 239 and a second portion 140, 240. In some examples, the first portion 139, 239 may be substantially straight and the second portion 140, 240 may be at least partly annular. The first portion 139, 239 may extend to the second portion 140, 240 such that the air flow may transition from the first portion 139, 239 to the second portion 140, 240 smoothly, thereby minimising air turbulence within the manifold 122, 222 that might otherwise affect the performance of the pod 110, 210. The first portion 139, 239 preferably extends to the second portion 140, 240 tangentially or at an angle with respect to the second portion 140, 240. The term “tangential” refers to an angle, such as an oblique angle, projecting from a part of the second portion 140, 240. In the example in which the second portion 140, 240 is at least partly annular, the first portion 139, 239 extends tangentially or at a tangent relative to a curved part of the second portion 140, 240.

Performance of the pod 110, 210 may be affected by turbulent air flow due to the unpredictability of the direction, speed, and pressure of air within the pod 110, 210. It is therefore preferable to avoid any features within the manifold 122, 222 which may cause additional turbulence, such as sharp turns and edges. It will be appreciated that the manifold 122, 222 shown in the figures is only one of a number of possible configurations. The first portion 139, 239 may, for example, include a gentle curve. A gentle curve may allow for manufacturing and assembly practicalities, where certain other features of the pod 110, 210 must be located in an optimal location for the first portion 139, 239 of the channel 138, 238.

The second side of the manifold 122, 222 may include a cavity 141, 241. Once the pod 110, 210 is assembled, the cavity 141, 241 partially defines a sonication chamber 142, 242 in which the mist is produced.

The base of the cavity 141, 241 includes a first aperture 145, 245. The first aperture 145, 245 extends through the centre of the annular channel 140, 240 in the first side of the manifold 122, 222. The first aperture 145, 245 and the centre of the annular channel 140, 240 thereby form a third section of the mist outlet conduit.

The base of the cavity 141, 241 also includes one or more further apertures 148, 248. The further apertures 148, 248 extend from the base of the cavity 141, 241 through to the second portion 140, 240 of the channel 138, 238 in the first side of the manifold 122, 222. The further apertures 148, 248 thereby allow air into the sonication chamber 142, 242 at an angle transverse to the atomisation surface of the ultrasonic transducer 123, 223. The air flow therefore contacts the ultrasonic transducer 123, 223 with an increased force and may result in more efficient aerosolization and/or mist extraction. In some examples, the air flow is substantially perpendicular to the atomisation surface.

Preferably, there are four further apertures 148, 248. In examples having two or more further apertures 148, 248, the further apertures 148, 248 are spaced, preferably evenly spaced, around the first aperture 145, 245. The air inlet flow and the mist outlet flow may therefore be coaxial.

The one or more further apertures 148, 248 of the air inlet conduit are preferably positioned radially inward of the edge of the atomisation surface. The apertures 148, 248 may be of any shape, including circular holes or elongate slots. The apertures 148, 248 are preferably of the same width and shape as the second portion 139, 239 of the channel 138, 238 so as to maximise air flow from the channel 138, 238 to the sonication chamber 142, 242.

In some examples, the second side of the manifold 122 may comprise protrusions 161, as shown in FIG. 7. In some examples, each protrusion 161 is a curved segment with the degree of curvature of the segment matching or substantially matching the degree of curvature of a part of an edge of the ultrasonic transducer 123. In some examples, there are four protrusions 161 in the form of quadrants that are positioned annularly and spaced apart from one another. The protrusions 161 may be positioned to contact the capillary 124 towards its outer edge. The protrusions 161 may therefore serve to keep the edge of the first portion of the capillary 124 in contact with the ultrasonic transducer 123, 223.

The protrusions 161 preferably extend around as much of the perimeter of the first portion of the capillary 124 as possible. Such a configuration reduces liquid leakage, and also controls the air flow through the manifold 122 to direct the air flow to the ultrasonic transducer 123. The gaps between the protrusions 161 therefore preferably only exist due to other features of the assembly, such as the further apertures 148 of the manifold 122 and the passage of the second portion(s) of the capillary 124. The protrusions 161 may further act to centre the manifold 122, and therefore the sonication chamber 142, over the ultrasonic transducer 123 during assembly.

Additionally or alternatively, a plurality of biasing elements 244 may extend from the base of the cavity 241 towards the second side of the manifold 222, as shown in FIGS. 14, 15, and 19. The biasing elements 244, are configured to urge the first portion of the capillary 224 into contact with the ultrasonic transducer 223 to enhance atomisation of the liquid. The biasing elements 244 may be of any size, shape and material. Preferably, there are four biasing elements 244. Even more preferably, the biasing elements 244 are evenly spaced across the surface of the capillary 224. The provision of the four biasing elements 244 achieves a more even spread of a biasing force that acts against the first portion of the capillary 224 than other examples that comprise fewer than four biasing elements 244. The even spread of the biasing force of the four biasing elements 244 ensures uniform contact between the first portion of the capillary 224 and the ultrasonic transducer 223. This optimises the transfer of ultrasonic waves generated by the ultrasonic transducer 223 to the liquid carried by the capillary 224, thereby helping to optimise the aerosolization of the liquid.

The capillary 124, 224 extends between the liquid channel 126, 226 of the liquid barrier wall 120, 220 and the sonication chamber 142, 242. In order to aid the passage of the capillary 124, 224, the manifold 122, 222 may include open slots 151, 251 in its side surfaces. In some examples, the slots 151, 251 may additionally or alternatively be positioned in the wall of the hollow interior of the spacer 121, 221. Any change in angle of a slot, indentation, or otherwise which is configured to receive a portion of the capillary 124, 224 may include a radius so as to not interfere with the fluid flow through the capillary 124, 224.

The pod 110, 210 further includes a lower body portion 152, 252 positioned between the spacer 121, 221 and the second end wall. The lower body portion 152, 252 has an upper surface, a lower surface, and at least one side extending therebetween. The lower surface may abut the second end wall of the housing 111, 211. The side(s) of the lower body portion 152 may, similar to the liquid barrier wall 120, have a contoured outer edge and a circumferential groove 163 such that the outer edge contacts the side wall in two distinct locations, thereby providing a double seal against the side wall. The upper surface may include dowels 153, 253 configured to locate in corresponding holes (not shown) in the lower surface of the spacer 121, 221. In some examples, the dowels 153, 253 are integrally formed within the lower body portion 152, 252. A through hole 154, 254 may extend through both the upper and lower surfaces of the lower body portion 152, 252. The through hole 154, 254 serves as a section of the air inlet conduit.

The lower body portion 152, 252 comprises a cavity 155, 255 configured to accept the transducer holder 150, 250. At least one, and preferably a plurality of passages, extend between the base of the cavity 155, 255 and the lower surface of the lower body portion 152, 252, the passages serving to allow electrical connections to pass therethrough. The lower body portion 152, 252 may include recesses around its periphery so that when the pod is assembled, the hooked elements 129, 229 are accommodated.

The transducer holder 150, 250 is sized and shaped to be accepted by the lower body portion 152, 252. The ultrasonic transducer 123, 223 is supported in position adjacent to and in communication with the sonication chamber 142, 242 by the transducer holder 150, 250. The transducer holder 150, 250 comprises a lower disc portion 156, 256, a gasket 130, 230, and an upper annular portion 157, 257, at least one of which may comprise a resiliently deformable material. The lower disc portion 156, 256 may be generally planar. The lower disc portion 156, 256 comprises holes through which electrical contacts 158, 258 may extend to enable the transfer of a signal to the ultrasonic transducer 123, 223. The lower disc portion 156, 256 further comprises an annular ridge to act as the supporting surface for the underside of the ultrasonic transducer 123, 223.

The upper annular portion 157, 257 is sized and shaped to contact the gasket 130, 230 and the spacer 121, 221. The gasket 130, 230 is preferably at least partially of silicone or another resiliently deformable material, and serves to seal the transducer holder 150, 250 to minimise liquid leakage between the lower disc portion 156, 256 and the upper annular portion 157, 257. The upper annular portion 157, 257 further acts to clamp the outer rim of the ultrasonic transducer 123, 223 between itself and the annular ridge of the lower disc portion 156, 256, via the gasket 130, 230, such that any vibrations are efficiently transferred to the capillary 124, 224, but preferably isolated from the housing 111, 211. The upper annular portion 157, 257 may incorporate a chamfer or radius on its inner edge, thereby aiding the change in direction of air flow within the sonication chamber 142, 242 whilst reducing turbulence. During assembly, the chamfer or radius may also interact with the protrusions 161 of the manifold 122 to aid in centring the manifold 122 and sonication chamber 142 over the ultrasonic transducer 123.

Further, the face of the upper annular portion 157, 257 which clamps the outer ring of the ultrasonic transducer 123, 223 may include an annular retaining ring 147, 247. The retaining ring 147, 247 may be of silicone or another plastic material, and acts to minimise energy loss by the ultrasonic transducer 123, 223 while still holding the ultrasonic transducer 123, 223 securely in position.

The components of the transducer holder 150, 250 may be attached to one another using heat staking (thermoplastic staking). In examples using heat staking to assemble the transducer holder 150, 250, one of the lower disc portion 156, 256 and the upper annular portion 157, 257 may comprise posts (not shown), with the other of the lower disc portion 156, 256 and the upper annular portion 157, 257, along with the gasket 130, 230, comprising holes for the posts to pass through. The silicone or other plastic material seals the components of the transducer holder 150, 250 together to minimise the risk of liquid flowing between the components of the transducer holder 150, 250 which might otherwise cause a malfunction.

The ultrasonic transducer 123, 223 is configured to convert an electrical input signal into high frequency vibrations. The atomisation surface of the ultrasonic transducer 123, 223 is adjacent to and in communication with the sonication chamber 142, 242 via the capillary 124, 224. In use, the atomisation surface is configured to turn the liquid, which saturates the capillary 124, 224, into a mist.

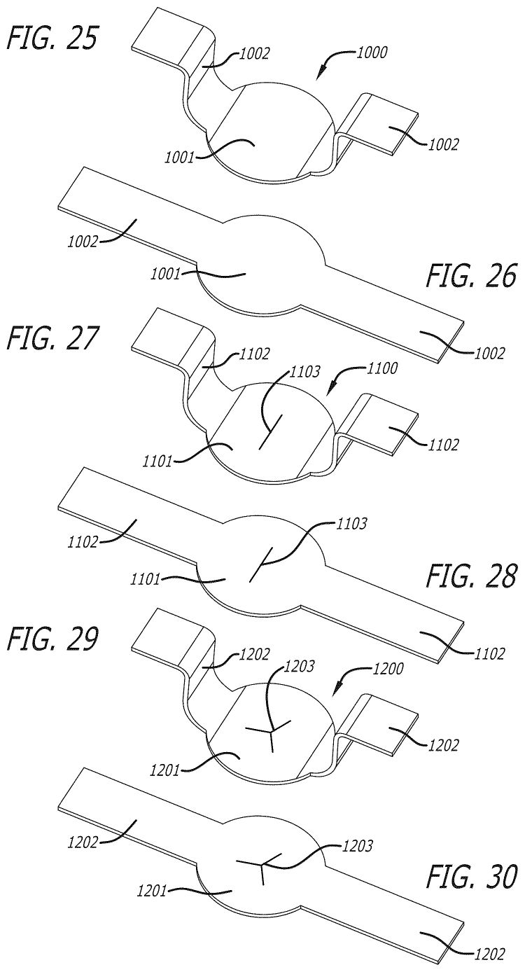

The capillary 124, 224, interchangeable with capillaries 1000, 1100, 1200, 1300, and 1400 of FIGS. 25 to 34, may be of any material capable of transporting liquid by capillary action. The shape of the capillary 124, 224 may be determined by the channel formed between the manifold 122, 222 and the spacer 121, 221, the channel formed between the spacer 121, 221 and the liquid barrier wall 120, 220, and/or the shape of the ultrasonic transducer 123, 223.

The capillary 1000 comprises a first portion 1001 and a second portion 1002. In some examples, the first portion 1001 is at least partly circular in shape to correspond with the shape of the ultrasonic transducer 123, 223. By “partly circular” it is meant that the first portion 1001 preferably includes at least one arcuate edge extending between the second portions 1002. In some examples, such as those depicted in the figures, two arcuate edges are provided, each arcuate edge extending from one of the second portions 1002 to another of the second portions 1002. In examples including more than one arcuate edge, they are preferably of the same length and/or radius, however this need not be the case. Uneven edges may be desired or necessary, for example, where a pod with is non-symmetrical. It is preferable that the first portion 1001 covers substantially all of the atomisation surface so as to maximise the surface area available for liquid to be atomised.

The second portion 1002 includes at least one, but preferably a plurality of arms which extend away from the first portion 1001. In examples employing a plurality of arms, the arms are preferable evenly spaced around the first portion 1001. In preferred examples, the arms are generally rectangular in shape, although their shape may depend on the design and configuration of the pod in which they are being implemented. For example, in the pods 110, 210 illustrated in FIGS. 2 and 14, the second portions 1002 extend upwards (i.e., transverse to the plane of the atomisation surface of the ultrasonic transducer 123, 223), and then radially outward with respect to the first portion 1001. The second portion 1002 may have any number of bends, as required by the design of the pod. In some embodiments the arms may be of differing lengths to one another. The first portion 1001 is at least partially superimposed on the atomisation surface of the ultrasonic transducer 123, 223, and preferably substantially covers the atomisation surface of the ultrasonic transducer 123, 223. The second portion 1002 is preferably adjacent the liquid outlet of the liquid channel 126, 226, and more preferably covers at least a portion of the liquid outlet. More preferably still, the second portion 1002 completely covers all liquid channels 126, 226 in the liquid barrier wall 120, 220. The liquid from the liquid chamber 125, 225 is therefore conducted from the liquid outlet of the liquid channel 126, 226 to the atomisation surface of the ultrasonic transducer 123, 223 by the capillary.

The first portion 1101 of the capillary 1100 may comprise an opening in the form of a slit 1103. A slit 1103 maximizes the surface area of the first portion 1101 of the capillary 1100, and therefore maximizes mist production, whilst allowing egress of bubbles from between the capillary 1100 and the ultrasonic transducer 123, 223. The slit 1103 may take any shape, and there may be any number of slits 1103, some examples of which are shown in FIGS. 28 to 32. Preferably, the slit 1103 passes through the centre of the first portion 1101 of the capillary 1100.

FIGS. 29 and 30 show an alternative capillary 1200. The capillary 1200 of this example includes three slits 1203. It is preferable that the slits 1203 join in the centre of the first portion 1201 of the capillary 1200, as this is where bubbles may escape most efficiently.

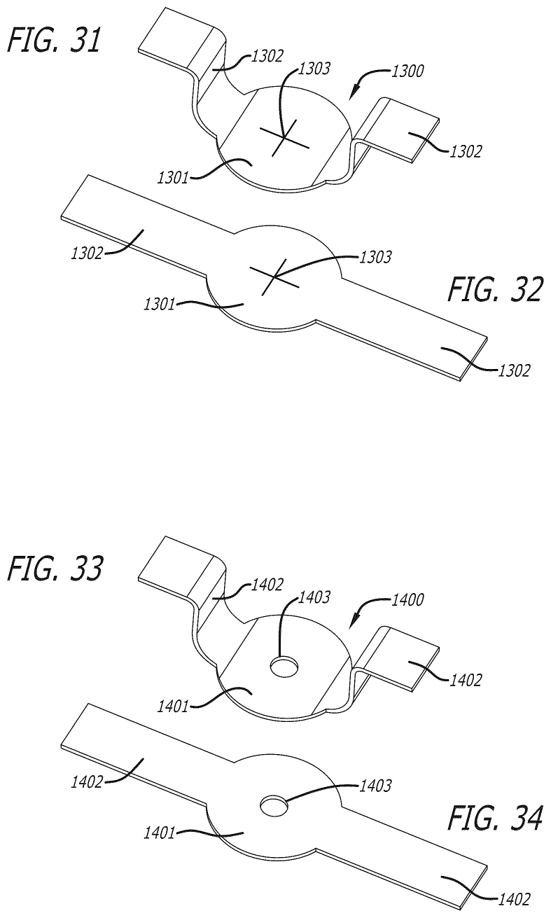

FIGS. 31 and 32 show a further alternative capillary 1300. The capillary 1300 of this example includes four slits 1303, although it can be readily seen that the slit pattern may be achieved by making only two slits. Again, it is preferable that the slits 1303 join in the centre of the first portion 1301 of the capillary 1300.

Alternatively, the opening may be a through hole 1403 as illustrated in FIGS. 33 and 34. The through hole 1403 is preferably centred in the first portion 1401 of the capillary 1400. In some examples, more than one through hole 1403 is provided. The at least one through hole 1403, similar to the slits referenced above, allows air bubbles to escape from between the capillary 1400 and the ultrasonic transducer 123, 223, and thereby promotes an improved contact between the atomization surface and the capillary, which increases the efficiency of mist production. In examples having more than one through hole 1403, the through holes 1403 are preferably spaced around the centre of the first portion 1401 of the capillary 1400.

It will be understood that where the capillary of any embodiment is of a woven material, an opening is defined as being larger than the holes naturally found in the weave of the fabric.

Further examples (not shown) may include slots instead of a through hole 1403 or a slit 1103, 1203, 1303. It will be appreciated that any number of each type of opening may be present on a capillary, and also that any of the described capillaries be interchangeable with any other capillary. Further still, the capillaries described above need not be used in combination with the pods of the present application, and the general principles may be used to incorporate a capillary into any pod.

In some examples, the capillary has a first portion with a diameter of approximately 6 mm to 10 mm, with 8 mm being preferred. Each of the second portions are preferably approximately 8 mm to 10 mm long, with 9.2 mm long being preferred and approximately 2 mm to 5 mm wide, with 3.6 mm wide being preferred. The capillary has a thickness of approximately 0.1 mm to 0.5 mm, with a 0.3 mm thickness being preferred.

When implemented into a pod, in some examples the second portions preferably each extend approximately 4 mm to 6 mm upwards, with 5 mm upwards being preferred, and approximately 3 mm to 5 mm radially, with 4 mm radially being preferred, or 7 mm to 11 mm radially, with 9 mm radially being preferred when measured from the centre of the first portion.

The configurations of the capillary above achieve an advantage of improved control of liquid flow to the atomisation surface of the ultrasonic transducer to which the capillary is applied. It is appreciated that the capillary could be scaled up or down in size and shape to correspond to the size and shape of the atomisation surface, whilst maintaining the similar size ratios between measurements of the first and second portions, and still achieve this effect.

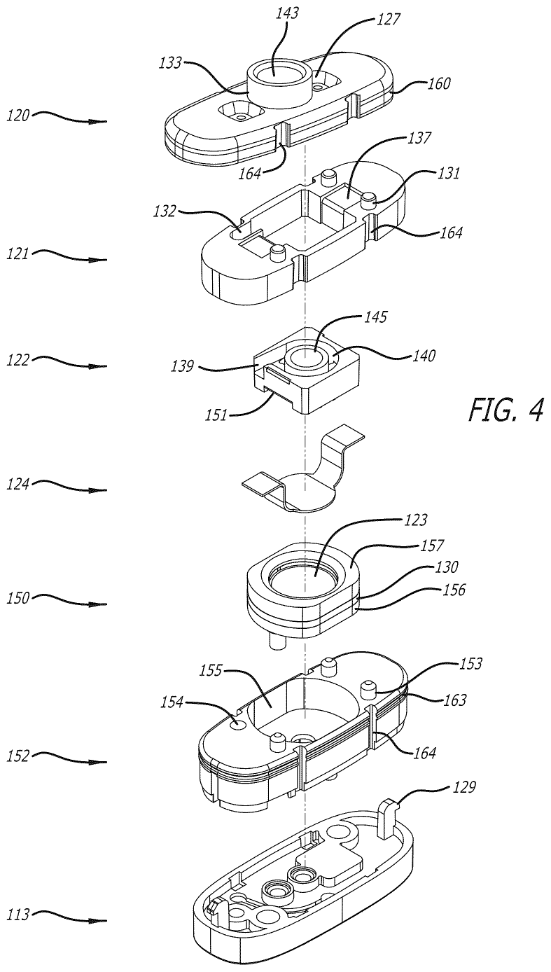

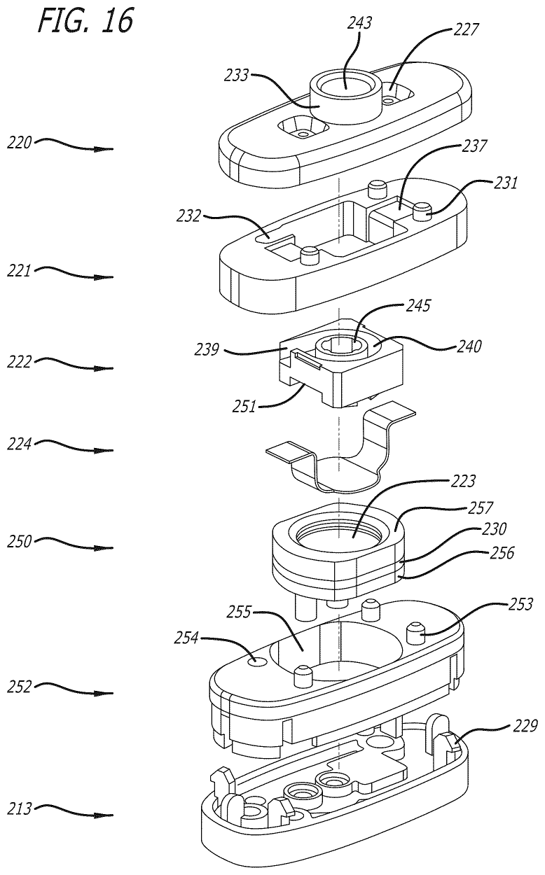

Referring now to FIGS. 4 and 16, which illustrate an exploded view of part of the pod 110, 210 and thus assists with the visualisation of the assembly. The transducer holder 150, 250 is placed within the cavity 155, 255 of the lower body portion 152, 252, and the spacer 121, 221 is coupled to the lower body portion 152, 252. The dowels 153, 253 of the lower body portion 152, 252 engage in holes on the lower surface of the spacer 121, 221. The dowels 153, 253 and holes may be any of a clearance, transitional, or interference fit, and serve to prevent excessive movement of the lower body portion 152, 252 relative to the spacer 121, 221. The through hole 154, 254 and the slot 132, 232 align so as to form a part of the air inlet conduit.

The capillary 124, 224 is inserted through the hollow interior of the spacer 121, 221 so that the first portion of the capillary 124, 224 is superimposed on the atomisation surface of the ultrasonic transducer 123, 223. The second portions of the capillary 124, 224 are positioned within the indentations 137, 237 in the spacer 121, 221.

The manifold 122, 222 is positioned within the hollow cavity of the spacer 121, 221, thereby forming the sonication chamber 142, 242. The protrusions 161 and/or biasing elements 244 urge the first portion of the capillary 124, 224 into contact with the atomisation surface of the ultrasonic transducer 123, 223. In examples utilising protrusions 161, said protrusions 161 may serve to centre the manifold 122 relative to the transducer holder 150. The second portion of the capillary 124, 224 passes through a channel formed between the manifold slots 151, 251 and the spacer 121, 221. The first portion 139, 239 of the manifold channel 138, 238 aligns with the slot 132, 232 in the spacer 121, 221, further defining the air inlet conduit.

The liquid barrier wall 120, 220 couples to the spacer 121, 221 by means of pegs 131, 231 on the spacer engaging with holes in the underside of the liquid barrier wall 120, 220. Similar to the dowels 153, 253 in the lower body portion 152, 252, the pegs 131, 231 may engage with their respective holes by means of a clearance, transitional, or interference fit, and serve to prevent excessive movement of the liquid barrier wall 120, 220 relative to the spacer 121, 221.

The second portion of the capillary 124, 224 is held in place within the indentations 137, 237 in the spacer 121, 221. Further, the slot 132, 232 in the spacer 121, 221, and both the first and second portions 139, 239, 140, 240 of the channel 138, 238 in the manifold 122, 222 are provided with a closing side by the liquid barrier wall 120, 220, the air inlet conduit thereby defined.

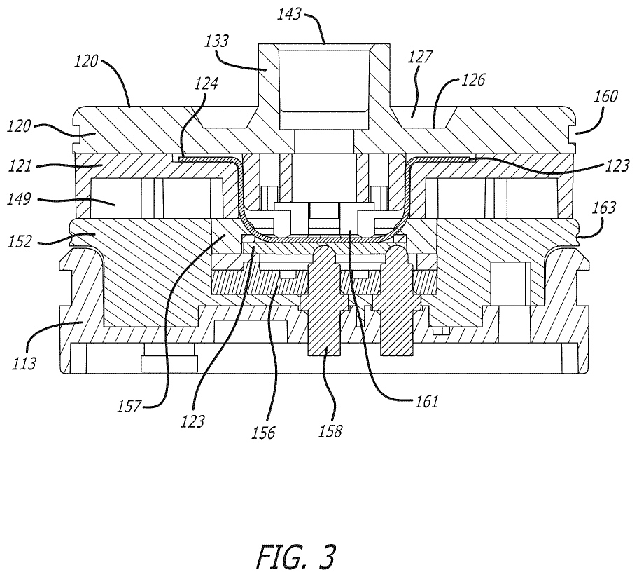

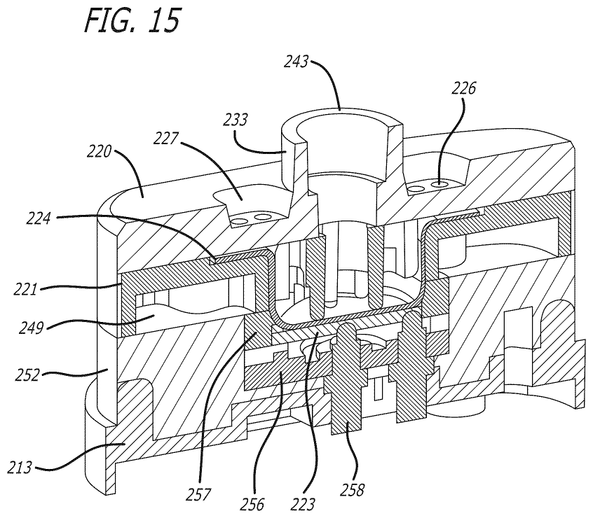

The end cap 113, 213, the lower body portion 152, 252, the transducer 123, 223 and transducer holder 150, 250, the spacer 121, 221, the capillary 124, 224, the manifold 122, 222 and the liquid barrier wall 120, 220 form the subassembly illustrated in FIGS. 3 and 15. The various slots and indentations interacting with the capillary 124, 224 may be shaped and/or sized in order to compress the capillary 124, 224 a predetermined amount. The compressive force on the capillary 124, 224 is low enough to avoid restricting liquid transfer, but high enough to prevent the liquid flooding the sonication chamber 142, 242. The channel formed by the various slots and indentations are preferably spaced and sized to fit the capillary 124, 224 without an air gap, so as to reduce leakage of the liquid and allow greater control of the flow of liquid being delivered to the sonication chamber 142, 242. Alternatively, the capillary 124, 224 may be shaped and sized to the design constraints of the components of the pod.

A collar 168 may be provided inside the central through hole 143 to encourage the seal between the liquid barrier wall 120 and the first portion of the mist outlet conduit 146. The collar 168 may also act as a reducer in examples where the outlet of the sonication chamber 142 and the first portion of the mist outlet conduit 146 are of a different diameter.

The abovementioned subassembly carries the advantage of being simple to manufacture, and also simple to assemble. For example, at least some of the various holes, channels, and protrusions are two dimensional forms, and not intricate and complex geometries. The various sections are thus efficient to manufacture using well established manufacturing techniques, such as machining, casting, and moulding. This also means that manufacture and assembly may be at least partially autonomous. The parts of the device may be assembled using automated robots on a production line with minimal human intervention. The device is therefore configured to be mass produced on a production line relatively easily and at low cost compared with conventional mist generator devices.

The subassembly may be positioned within the housing 111, 211 of the pod 110, 210 such that the first section of the mist outlet conduit 146, 246 is inserted into the central through hole 143, 243 in the liquid barrier wall 120, 220. The inner edge of the central protrusion 133, 233 may be chamfered in order to aid insertion. The central protrusion 133, 233 may have a stepped diameter and therefore act as a stop against the first portion 146, 246 of the mist outlet conduit.

The end cap 113, 213 may include a plurality of holes to provide continuity of the holes in the lower body portion 152, 252. For example, the end cap 113, 213 may include an air inlet hole 136, 236, preferably configured to align with the through hole 154, 254 which forms a portion of the air inlet conduit.

The absorbing element 128, 228 and the mouthpiece 112, 212 are coupled at the first axial end of the housing 111, 211 to form the pod 110, 210, example configurations of which can be seen in of FIGS. 2 and 14.

The above-described pod 110, 210 assembly comprises both an air-tight air inlet conduit and an air-tight mist outlet conduit, each formed of multiple components of the pod 110, 210. Air may be conducted to the sonication chamber 142, 242 from proximate the second end wall via the through hole 154, 254 in the lower body portion 152, 252, the slot 132, 232 formed in the spacer 121, 221, the channel formed in the manifold 122, 222, and through the one or more inlet apertures 148, 248. After combining with the liquid particles, the mist exits the pod 110, 210 through the first aperture 145, 245 in the manifold 122, 222, the first portion of the mist outlet conduit 146, 246, the absorbent element 128, 228, and the mouthpiece outlet port 117, 217. The mist outlet conduit preferably passes through the liquid chamber 125, 225.

Although the assembly has been described in a certain order, it will be appreciated that this is only an example and the components may be assembled in any plausible order. Similarly, terms such as “upper”, “lower”, and “side” are not to be construed as limiting, but for ease of reference to the figures.

The above-described pod 110, 210 is configured to be coupled to a driver, the driver comprising the means for powering and, in some embodiments, controlling the pod 110, 210. The pod 110, 210 is typically at least partially received by an axial end of the driver, and more specifically a cavity in the driver. The pod 110, 210, and more preferably the end cap 113, 213, may include a seal around its lower edge to seal against the driver.

The end cap 113, 213 may comprise retention pins 135, 235 (seen in FIGS. 9 and 21) for mounting a printed circuit board (PCB) to the pod 110, 210. In some examples, a PCB is mounted to the pod 110, 210 to sit at least partly in the recess in the base of the pod 110, 210. A microchip may be carried by the PCB and positioned so that the microchip sits on the surface of the PCB and is positioned within a further recess in the pod 110, 210 when the PCB is mounted to the pod 110, 210. In some examples, the microchip is a one-time-programmable integrated circuit (OTP IC) that may be used to identify and/verify the authenticity of the pod 110, 210.

In some examples, the retention pins 135, 235 are magnetic, and are configured to magnetically couple the driver to the pod 110, 210. In some examples, the retention pins 135, 235 are magnets that are mounted to the pod 110, 210 and arranged so that the ends of the retention pins 135, 235 have opposite polarities to one another. In these examples, the polarities of the magnetic retention pins 135, 235 must match the polarities of corresponding magnets provided on a driver so that the pod 110, 210 can only be coupled to the driver in one orientation. The magnetic pins 135, 235 repel the magnets on the driver when the pod 110, 210 is moved towards the driver in an incorrect orientation. This ensures that a user couples the pod 110, 210 to the driver in the correct orientation when replacing the pod 110, 210.

The driver may house an electrical storage device configured to power the pod 110, 210 so that the driver generates a drive signal which drives the ultrasonic transducer 123, 223 within the pod 110, 210. The electrical storage device can be a battery, including but not limited to a lithium-ion, alkaline, zinc-carbon, nickel-metal hydride, or nickel-cadmium battery; a super capacitor; or a combination thereof. The electrical storage device may be rechargeable. In examples utilizing a rechargeable electrical storage device, a charging port may be provided so that the electrical storage device does not need to be removed from the driver. The electrical storage device may be primarily selected to deliver a constant voltage independent of charge level. Otherwise, the performance may degrade over time. Preferred electrical storage devices that are able to provide a consistent voltage output over the life of the device include lithium-ion and lithium polymer batteries.

Electrical communication between the driver and the pod 110, 210 may be established using electrical contacts 158, 258.

A circuit board may carry at least one integrated circuit and/or a microprocessor. In some arrangements, the at least one integrated circuit and/or the microprocessor is configured to process data from a sensor which senses a parameter indicative of the operation of the ultrasonic transducer 123, 223 and controls the driver to vary the drive signal output to the ultrasonic transducer 123, 223 in a feedback loop.

In some arrangements, the pod 110, 210 or driver comprises an activation sensor which detects when the user draws on the mouthpiece 112, 212 and activates the ultrasonic transducer 123, 223 to generate a mist. The activation sensor can be selected to detect changes in pressure, air flow, or vibration. In one arrangement, the activation sensor is a pressure sensor.

In some arrangements, the integrated circuit comprises a frequency controller which is configured to control the frequency of the drive signal output from the driver to the ultrasonic transducer 123, 223. The frequency controller comprises a processor and a memory, the memory storing executable instructions which, when executed by the processor, cause the processor to perform at least one function of the frequency controller.

In some arrangements, the driver drives the ultrasonic transducer 123, 223 with a signal having a frequency of 2.8 MHz to 3.2 MHz in order to atomize a liquid having a liquid viscosity of 1.05 Pa.s to 1.412 Pa.s. Such a frequency enables the ultrasonic transducer 123, 223 to produce a bubble volume of about 0.25 to 0.5 microns. However, for liquids with a different viscosity or for other applications the ultrasonic transducer 123, 223 may be driven at a different frequency. Parameters affecting the optimal frequency include the transducer manufacturing process and tolerances, the physical load on the transducer, the local and ambient temperature, and the distance from the transducer to the power source.

The driver may have a wireless communication system, such as in the form of a Bluetooth Low Energy capable microcontroller. The wireless communication system is in communication with the at least one integrated circuit and/or the microprocessor of the device and is configured to transmit and receive data between the driver and a computing device, such as a smartphone.

The connectivity via Bluetooth Low Energy to a companion mobile application allows for remote control of the mist inhalation device.

The apparatus described above may comprise an identification arrangement that allows only genuine pods 110, 210 from the manufacturer to be used with the driver. This anti-counterfeiting measure may be implemented in the pod 110, 210 as a specific custom integrated circuit (IC) that is bonded to the pod 110, 210. The IC contains truly unique information that allows complete traceability of the pod 110, 210 (and its contents) over its lifetime as well as a precise monitoring of the consumption by the user. The IC allows the pod 110, 210 to function and to generate mist only when authorized. The unique information can be read by the mist inhalation device to ascertain information such as whether the pod 110, 210 is a genuine and/or certified pod, and whether the pod has been previously fully discharged, and therefore possibly refilled with counterfeit liquid. If certain conditions are met or not met, then the at least one integrated circuit and/or the microprocessor of the driver may allow or prevent the use of the pod 110, 210 with the driver.

In examples where the pod 110, 210 has an air inlet port in a position which is covered by the driver housing, the driver may include a conduit to convey air from the surroundings to the air inlet port of the pod 110, 210.

It will be appreciated that the pod may be used in mist inhalation devices different to those disclosed, and vice versa. In such examples, the air inlet seal and the mist outlet seal are ruptured, the capillary conducts the liquid from the liquid chamber to the atomisation surface to be atomised at the atomisation surface, and the mist generated is conducted through the mist outlet conduit to the mist outlet port.

When used in this specification and the appended claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or to encompass equivalents.

1. A mist inhalation pod for use with a driver, the pod comprising:
a housing having a first end, an opposite second end and at least one side wall extending between the first end and the second end;
a first end wall proximate the first end and the side wall, the first end wall closing the first end of the housing, the first end wall being provided with a mist outlet port;
a second end wall proximate the second end and the side wall, the second end wall closing the second end of the housing;
a liquid barrier wall positioned within the housing and spaced apart from the first end wall, the liquid barrier wall extending towards the side wall of the housing to form a liquid seal between the liquid barrier wall and the side wall, the liquid barrier wall having at least one liquid channel with a liquid inlet and a liquid outlet;
a liquid chamber defined by the liquid barrier wall, the first end wall and the side wall of the housing, the liquid chamber containing a liquid to be atomised, the liquid comprising a therapeutic, the liquid inlet being in liquid communication with the liquid chamber for conducting the liquid through the liquid outlet;
a spacer positioned within the housing between the liquid barrier wall and the second end wall, the spacer having a perimeter extending toward the side wall of the housing, the spacer including a hollow interior surrounded by the perimeter;
a fluid flow manifold positioned at least partially within the hollow interior of the spacer and including a first side proximate the first end wall, and a second side proximate the second end wall, the first side of the fluid flow manifold including a channel having a first portion and a second portion, the first portion extending to the second portion tangentially, and the second side of the fluid flow manifold having a cavity, the cavity including a first aperture for allowing fluid flow in a first direction, and one or more further apertures for allowing fluid flow in a second direction;
a sonication chamber including the cavity of the fluid flow manifold;
an ultrasonic transducer positioned between the sonication chamber and the second end wall, the ultrasonic transducer having an atomisation surface adjacent to the sonication chamber and in communication with the sonication chamber;
a capillary having a first portion at least partly superimposed on the atomisation surface of the ultrasonic transducer and a second portion adjacent to the liquid outlet of the liquid channel, wherein the second portion of the capillary covers at least a portion of the liquid outlet and is configured to conduct the liquid from the liquid outlet to the atomisation surface to generate a mist;
an air inlet conduit, including the one or more further apertures in the fluid flow manifold, forming an air-tight channel for conducting air through the spacer and along the channel in the first side of the fluid flow manifold, the air inlet conduit extending from proximate the second end wall, through the spacer and through the fluid flow manifold to the sonication chamber, a first end of the air inlet conduit being in fluid communication with an air inlet port proximate the second end wall of the housing and a second end of the air inlet conduit being in fluid communication with the sonication chamber; and
a mist outlet conduit, including the first aperture in the fluid flow manifold, forming an air-tight channel for conducting the mist through the liquid chamber and any liquid contained therein, the mist outlet conduit extending from the first end wall, through the liquid chamber, through the liquid barrier wall, and through the fluid flow manifold to the sonication chamber, a first end of the mist outlet conduit being in fluid communication with a mist outlet port in the first end wall of the housing and a second end of the mist outlet conduit being in fluid communication with the sonication chamber,
wherein, in use, the capillary conducts the liquid from the liquid chamber to the atomisation surface to be atomised, and the mist generated is conducted through the mist outlet conduit to the mist outlet port.

2. The mist inhalation pod of claim 1, wherein the first portion of the channel is substantially straight between the spacer and the second portion of the channel.

3. The mist inhalation pod of claim 1, wherein the second portion of the channel is annular, the annular second portion of the channel surrounding the first aperture of the fluid flow manifold.

4. The mist inhalation pod of claim 1, wherein the one or more further apertures include at least four apertures, the at least four apertures being spaced around the first aperture, the fluid flow in the first direction thereby being coaxial to the fluid flow in the second direction.

5. The mist inhalation pod of claim 1, wherein the one or more further apertures are slots of the same width and shape as the second portion of the channel.

6. The mist inhalation pod of claim 1, wherein the one or more further apertures of the air inlet conduit are positioned radially inward of the first portion of the capillary.

7. The mist inhalation pod of claim 6, wherein air enters the sonication chamber transverse to the atomisation surface of the ultrasonic transducer.

8. The mist inhalation pod of claim 1, further comprising a mouthpiece positioned proximate the first end of the housing, the mouthpiece including a mist inhalation port in fluid communication with the mist outlet port in the housing.

9. A mist inhalation pod for use with a driver, the pod comprising:
a housing having a first end, an opposite second end and at least one side wall extending between the first end and the second end;
a first end wall proximate the first end and the side wall, the first end wall closing the first end of the housing, the first end wall being provided with a mist outlet port;
a second end wall proximate the second end and the side wall, the second end wall closing the second end of the housing;
a liquid barrier wall positioned within the housing and spaced apart from the first end wall, the liquid barrier wall having at least one liquid channel with a liquid inlet and a liquid outlet;
a liquid chamber partially defined by the liquid barrier wall, the liquid chamber containing a liquid to be atomised, the liquid comprising a therapeutic, the liquid inlet being in liquid communication with the liquid chamber for conducting the liquid through the liquid outlet;
a spacer positioned within the housing between the liquid barrier wall and the second end wall, the spacer including a hollow interior surrounded by a perimeter;
a fluid flow manifold positioned at least partially within the hollow interior of the spacer and including a first side proximate the first end wall, and a second side proximate the second end wall, the first side of the fluid flow manifold including a channel having a first portion and a second portion, the first portion extending to the second portion tangentially, and the second side of the fluid flow manifold having a cavity, the cavity including a first aperture for allowing fluid flow in a first direction, and one or more further apertures for allowing fluid flow in a second direction;
a sonication chamber including the cavity of the fluid flow manifold;
an air inlet conduit for conducting air to the sonication chamber; and
a mist outlet conduit for conducting mist to the mist outlet port,
wherein, in use, the liquid is conducted from the liquid chamber to the sonication chamber to be atomised, and the mist generated is conducted through the mist outlet port.

10. The mist inhalation pod of claim 9, wherein the first portion of the channel is substantially straight between the spacer and the second portion of the channel.

11. The mist inhalation pod of claim 9, wherein the second portion of the channel is annular, the annular second portion of the channel surrounding the first aperture in the fluid flow manifold.

12. The mist inhalation pod of claim 9, wherein the one or more further apertures include at least four apertures, the at least four apertures being spaced around the first aperture, the fluid flow in the first direction thereby being coaxial to the fluid flow in the second direction.

13. The mist inhalation pod of claim 9, wherein the one or more further apertures are slots of the same width and shape as the second portion of the channel.

14. The mist inhalation pod of claim 9, wherein the one or more further apertures of the air inlet conduit are positioned radially inward of the first portion of the capillary.

15. The mist inhalation pod of claim 14, wherein air enters the sonication chamber transverse to the atomisation surface of the ultrasonic transducer.

16. The mist inhalation pod of claim 9, further comprising a mouthpiece positioned proximate the first end of the housing, the mouthpiece including a mist inhalation port in fluid communication with the mist outlet port in the housing.

17. The mist inhalation pod of claim 9, further comprising an ultrasonic transducer positioned between the sonication chamber and the second end wall, the ultrasonic transducer having an atomisation surface adjacent to the sonication chamber and in communication with the sonication chamber.

18. The mist inhalation pod of claim 9, wherein the liquid barrier wall extends towards the side wall of the housing to form a liquid seal between the liquid barrier wall and the side wall.

19. A mist inhalation device comprising a pod and a driver,
the pod comprising:
a housing having a first end, an opposite second end and at least one side wall extending between the first end and the second end;
a first end wall proximate the first end and the side wall, the first end wall closing the first end of the housing, the first end wall being provided with a mist outlet port;
a second end wall proximate the second end and the side wall, the second end wall closing the second end of the housing;
a liquid barrier wall positioned within the housing and spaced apart from the first end wall, the liquid barrier wall having at least one liquid channel with a liquid inlet and a liquid outlet;
a liquid chamber partially defined by the liquid barrier wall, the liquid chamber containing a liquid to be atomised, the liquid comprising a therapeutic, the liquid inlet being in liquid communication with the liquid chamber for conducting the liquid through the liquid outlet;
a spacer positioned within the housing between the liquid barrier wall and the second end wall, the spacer including a hollow interior surrounded by a perimeter;
a fluid flow manifold positioned at least partially within the hollow interior of the spacer and including a first side proximate the first end wall, and a second side proximate the second end wall, the first side of the fluid flow manifold including a channel having a first portion and a second portion, the first portion extending to the second portion tangentially, and the second side of the fluid flow manifold having a cavity, the cavity including a first aperture for allowing fluid flow in a first direction, and one or more further apertures for allowing fluid flow in a second direction;
a sonication chamber including the cavity of the fluid flow manifold;
an air inlet conduit for conducting air to the sonication chamber; and
a mist outlet conduit for conducting mist to the mist outlet port,
wherein, in use, the liquid is conducted from the liquid chamber to the sonication chamber to be atomised, and the mist generated is conducted through the mist outlet port, and
the driver including:
a housing having a cavity configured to receive at least part of the pod; and
driver circuitry for supplying a drive signal to the pod.

20. The mist inhalation device of claim 19, wherein the driver housing includes a hole, and the driver includes a conduit extending from the hole to the pod, the conduit configured to convey air from the surroundings to the air inlet conduit of the pod.
